(19) United States
(12) Patent Application Publication
(10) Pub. No.: US 2024/0180950 A1
(43) Pub. Date: Jun. 6, 2024

(54) Prevention and Treatment of Post-Infection Neurological Disorders and Clotting/Thrombotic Disorders

(71) Applicant: Steven BARANOWITZ, Wyncote, PA (US)

(72) Inventor: Steven BARANOWITZ, Wyncote, PA (US)

(21) Appl. No.: 18/568,945

(22) PCT Filed: Jun. 14, 2022
(86) PCT No.: PCT/US2022/033325

§ 371 Date: Dec. 11, 2023

Related U.S. Application Data
(60) Provisional application No. 63/210,516, filed on Jun. 15, 2021.

(51) Int. Cl. A61K 31/708; A61K 9/00

(52) U.S. Cl. CPC A61K 31/708; A61K 9/0053; A61K 9/0095

(57) ABSTRACT

Compositions and methods for the prevention and treatment of signs and symptoms of post-viral neurologic disorders, such as those that occur in COVID 19.

### CROSS-REFERENCE TO RELATED APPLICATION

This international application claims benefit of U.S. Ser. No. 63/210,516 filed Jun. 15, 2021 and U.S. Ser. No. 63/263,581 filed Nov. 5, 2021, the entireties of which is incorporated herein by reference.

### SPECIFICATION

### Background

The inventor has discovered that the mechanistic cause of many neurological disorders and clotting/thrombotic disorders, including those occurring during and after infection with COVID-19 and other viruses, as well as other infectious agents such as bacteria, fungi, and parasites, is the depletion of nucleosides and nucleotides, especially guanosine and its derivatives. Therefore, signs and symptoms in post-viral neurologic disorders such as those that occur in COVID 19, and in other post-infectious syndromes, and in clotting/thrombotic disorders which occur during and after infection, and especially in coronavirus “long hauler” patients, include brain fog, fatigue, lethargy, tremors, cognitive dysfunction, psychological and psychiatric disorders and illnesses, and clotting/thrombotic illnesses including stoke, myocardial infarction, deep vein thrombosis, pulmonary embolism, and other disorders of hemostasis, result from the relative deficit of nucleosides and nucleotides, especially guanosine and its derivatives in the central and peripheral nervous systems and the hematologic and cardiovascular systems.

It is well known that infection can dramatically increase the risk of clotting disorder (Beristain-Covarrubias N, Perez-Toledo M, Thomas M R, Henderson I R, Watson S P, Cunningham A F. 2019. Understanding Infection-Induced Thrombosis: Lessons Learned From Animal Models. Front Immunol 10:2569), and these disorders entail massive unmet medical needs. For example, stroke is one of the top causes of death in the United States (Centers for Disease Control and Prevention. Leading causes of death. https://www.cdc.gov/nchs/fastats/leading-causes-of-death.htm Accessed10/31/2021.).

There are a wide range of clotting thrombotic disorders, and extensive examples can be found in Thrombosis and Hemorrhage 3rd Edition. 2002, Joseph Loscalzo (Editor), Andrew I. Schafer (Editor), Lippincott Williams & Wilkins, Philadelphia.

Infectious agents such as viruses, bacteria, fungi, and parasites require nucleotides to reproduce their genetic material so the viruses or organisms can multiply. For instance, both DNA and RNA viruses, such as SARS-CoV-2, require guanosine and its derivatives to construct their genetic material when replicating. These infectious organisms obtain their nucleotides from host cells including cells in the nervous system, thus depleting them of nucleosides and nucleotides and especially guanosine and its derivatives.

Furthermore, the inventor has discovered that these signs and symptoms can be mitigated or cured by supplementing the body's pool of nucleosides and nucleotides, especially guanosine and its derivatives, by oral, enteral or parenteral means.

Additionally, these signs and symptoms can be prevented by supplementing the body's pool of nucleosides and nucleotides, especially guanosine and its derivatives, by oral, enteral or parenteral means during the infectious episode.

The inventor has discovered that the mechanistic cause of signs and symptoms of neurological disorders and clotting/thrombotic disorders which occur in patients with autoimmune diseases such as lupus, are similarly due to nucleoside and nucleotide depletion, especially the depletion of guanosine and its derivatives by anti-guanosine antibodies present in such patients. The inventor has discovered that these signs and symptoms can be mitigated or cured by supplementing the body's pool of nucleosides and nucleotides, especially guanosine and its derivatives, by oral, enteral or parenteral means.

All references cited herein are incorporated herein by reference in their entireties.

### BRIEF SUMMARY

The disclosure provides a method of preventing and/or treating a post-pathogenic infection neurological syndrome in a patient, the method comprising the steps of: selecting a patient in need of preventing and/or treating a post-pathogenic infection neurological syndrome; administering to the patient a nucleoside and/or nucleotide formulation which is an oral, enteral, or parenteral nutritional or pharmaceutical formulation; wherein the post-pathogenic infection neurological syndrome is prevented and/or treated in the patient. The disclosure provides a method of preventing and/or treating a post-pathogenic infection neurological syndrome in a patient as disclosed herein in a patient wherein the nucleoside and/or nucleotide formulation is an oral formulation. The disclosure provides a method of preventing and/or treating a post-pathogenic infection neurological syndrome in a patient as disclosed herein wherein the nucleoside and/or nucleotide formulation is an enteral formulation. The disclosure provides a method of preventing and/or treating a post-pathogenic infection neurological syndrome in a patient as disclosed herein wherein the nucleoside and/or nucleotide formulation is a parenteral formulation. The disclosure provides a method of preventing and/or treating a post-pathogenic infection neurological syndrome in a patient as disclosed herein wherein the nucleoside and/or nucleotide formulation comprises nucleotides and/or nucleotide precursors selected from the group consisting of nucleosides, purine bases, pyrimidine bases, ribose, and deoxyribose. The disclosure provides a method of preventing and/or treating a post-pathogenic infection neurological syndrome in a patient as disclosed herein wherein the nucleotide is in monophosphate, diphosphate, or triphosphate form. The disclosure provides a method of preventing and/or treating a post-pathogenic infection neurological syndrome in a patient as disclosed herein wherein the nucleotide may be a ribonucleotide or a deoxyribonucleotide. The disclosure provides a method of preventing and/or treating a post-pathogenic infection neurological syndrome in a patient as disclosed herein wherein the nucleotides may be monomeric, dimeric, or polymeric (including RNA and DNA). The disclosure provides a method of preventing and/or treating a post-pathogenic infection neurological syndrome in a patient as disclosed herein wherein the nucleoside and/or nucleotide may be present in the nutritional composition as a free acid or in the form of a salt, preferably a monosodium salt. The disclosure provides a method of preventing and/or treating a post-pathogenic infection neurological syndrome in a patient as disclosed herein wherein the nucleotide is selected from the group consisting of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, inosine 5′-monophosphate, and combinations thereof. The disclosure provides a method of preventing and/or treating a post-pathogenic infection neurological syndrome in a patient as disclosed herein wherein the nucleoside and/or nucleotide formulation is a guanosine formulation. The disclosure provides a method of preventing and/or treating a post-pathogenic infection neurological syndrome in a patient as disclosed herein wherein the nucleoside and/or nucleotide formulation is administered to provide about 50 mg to about 999 mg of guanosine to the patient. The disclosure provides a method of preventing and/or treating a post-pathogenic infection neurological syndrome in a patient as disclosed herein wherein the nucleoside and/or nucleotide formulation is administered in 2 or 3 divided doses. The disclosure provides a method of preventing and/or treating a post-pathogenic infection neurological syndrome in a patient as disclosed herein wherein the nucleoside and/or nucleotide formulation is administered in doses selected from the group consisting of 250 mg, 500 mg, 1000 mg, 2000 mg, and 4000 mg. The disclosure provides a method of preventing and/or treating a post-pathogenic infection neurological syndrome in a patient as disclosed herein wherein the nucleoside and/or nucleotide formulation is administered in single or divided doses of one to four times daily. The disclosure provides a method of preventing and/or treating a post-pathogenic infection neurological syndrome in a patient as disclosed herein wherein the nucleoside and/or nucleotide formulation is administered multiple times per day. The disclosure provides a method of preventing and/or treating a post-pathogenic infection neurological syndrome in a patient as disclosed herein wherein the nucleoside and/or nucleotide formulation is administered once per day, twice per day, three times per day, four times per day, or five times per day. The disclosure provides a method of preventing and/or treating a post-pathogenic infection neurological syndrome in a patient as disclosed herein wherein the pathogenic infection is selected from the group consisting of viral infection, bacterial infection, fungal infection, parasitic infection, and combinations thereof. The disclosure provides a method of preventing and/or treating a post-pathogenic infection neurological syndrome in a patient as disclosed herein wherein the pathogenic infection is a viral infection. The disclosure provides a method of preventing and/or treating a post-pathogenic infection neurological syndrome in a patient as disclosed herein wherein the viral infection is selected from the group consisting of coronavirus, SARS, SARS-Cov2, Zika virus, Norovirus, Respiratory Syncytial Virus, Influenza, Adenovirus 5, HPV 11, Lassa Fever virus, Powassan virus, Rift Valley virus, West Nile virus, and combinations thereof.

The disclosure provides a method of preventing and/or treating a post-pathogenic infection clotting/thrombotic disorder in a patient, the method comprising the steps of: selecting a patient in need of preventing and/or treating a post-pathogenic infection clotting/thrombotic disorder; administering to the patient a nucleoside and/or nucleotide formulation which is an oral, enteral, or parenteral nutritional or pharmaceutical formulation; wherein the post-pathogenic infection clotting/thrombotic disorder is prevented and/or treated in the patient. The disclosure provides a method of preventing and/or treating a post-pathogenic infection clotting/thrombotic disorder in a patient wherein the nucleoside and/or nucleotide formulation is an oral formulation. The disclosure provides a method of preventing and/or treating a post-pathogenic infection clotting/thrombotic disorder in a patient wherein the nucleoside and/or nucleotide formulation is an enteral formulation. The disclosure provides a method of preventing and/or treating a post-pathogenic infection clotting/thrombotic disorder in a patient wherein the nucleoside and/or nucleotide formulation is a parenteral formulation. The disclosure provides a method of preventing and/or treating a post-pathogenic infection clotting/thrombotic disorder in a patient wherein the nucleoside and/or nucleotide formulation comprises nucleotides and/or nucleotide precursors selected from the group consisting of nucleosides, purine bases, pyrimidine bases, ribose, and deoxyribose. The disclosure provides a method of preventing and/or treating a post-pathogenic infection clotting/thrombotic disorder in a patient wherein the nucleotide is in monophosphate, diphosphate, or triphosphate form. The disclosure provides a method of preventing and/or treating a post-pathogenic infection clotting/thrombotic disorder in a patient wherein the nucleotide may be a ribonucleotide or a deoxyribonucleotide. The disclosure provides a method of preventing and/or treating a post-pathogenic infection clotting/thrombotic disorder in a patient wherein the nucleotides may be monomeric, dimeric, or polymeric (including RNA and DNA). The disclosure provides a method of preventing and/or treating a post-pathogenic infection clotting/thrombotic disorder in a patient wherein the nucleoside and/or nucleotide may be present in the nutritional composition as a free acid or in the form of a salt, preferably a monosodium salt. The disclosure provides a method of preventing and/or treating a post-pathogenic infection clotting/thrombotic disorder in a patient wherein the nucleotide is selected from the group consisting of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, inosine 5′-monophosphate, and combinations thereof. The disclosure provides a method of preventing and/or treating a post-pathogenic infection clotting/thrombotic disorder in a patient wherein the nucleoside and/or nucleotide formulation is a guanosine formulation. The disclosure provides a method of preventing and/or treating a post-pathogenic infection clotting/thrombotic disorder in a patient wherein the nucleoside and/or nucleotide formulation is administered to provide about 50 mg to about 999 mg of guanosine to the patient. The disclosure provides a method of preventing and/or treating a post-pathogenic infection clotting/thrombotic disorder in a patient wherein the nucleoside and/or nucleotide formulation is administered in 2 or 3 divided doses. The disclosure provides a method of preventing and/or treating a post-pathogenic infection clotting/thrombotic disorder in a patient wherein the nucleoside and/or nucleotide formulation is administered in doses selected from the group consisting of 250 mg, 500 mg, 1000 mg, 2000 mg, and 4000 mg. The disclosure provides a method of preventing and/or treating a post-pathogenic infection clotting/thrombotic disorder in a patient wherein the nucleoside and/or nucleotide formulation is administered in single or divided doses of one to four times daily. The disclosure provides a method of preventing and/or treating a post-pathogenic infection clotting/thrombotic disorder in a patient wherein the nucleoside and/or nucleotide formulation is administered multiple times per day. The disclosure provides a method of preventing and/or treating a post-pathogenic infection clotting/thrombotic disorder in a patient wherein the nucleoside and/or nucleotide formulation is administered once per day, twice per day, three times per day, four times per day, or five times per day. The disclosure provides a method of preventing and/or treating a post-pathogenic infection clotting/thrombotic disorder in a patient wherein the pathogenic infection is selected from the group consisting of viral infection, bacterial infection, fungal infection, parasitic infection, and combinations thereof. The disclosure provides a method of preventing and/or treating a post-pathogenic infection clotting/thrombotic disorder in a patient wherein the pathogenic infection is a viral infection. The disclosure provides a method of preventing and/or treating a post-pathogenic infection clotting/thrombotic disorder in a patient wherein the viral infection is selected from the group consisting of coronavirus, SARS, SARS-Cov2, Zika virus, Norovirus, Respiratory Syncytial Virus, Influenza, Adenovirus 5, HPV 11, Lassa Fever virus, Powassan virus, Rift Valley virus, West Nile virus, and combinations thereof. The disclosure provides a method of preventing and/or treating a post-pathogenic infection clotting/thrombotic disorder in a patient wherein said disease or disorder is a thrombotic disease or disorder and/or involves a blood clot thrombus or the potential formation of a blood clot thrombus. The disclosure provides a method of preventing and/or treating a post-pathogenic infection clotting/thrombotic disorder in a patient wherein said thrombotic disease or disorder comprises acute coronary syndrome, thromboembolism, and/or thrombosis. The disclosure provides a method of preventing and/or treating a post-pathogenic infection clotting/thrombotic disorder in a patient wherein said thromboembolism comprises venous thromboembolism, arterial thromboembolism, and/or cardiogenic thromboembolism. The disclosure provides a method of preventing and/or treating a post-pathogenic infection clotting/thrombotic disorder in a patient wherein said venous thromboembolism comprises deep vein thrombosis and/or pulmonary embolism. The disclosure provides a method of preventing and/or treating a post-pathogenic infection clotting/thrombotic disorder in a patient wherein said thrombotic disease or disorder involves dysfunctional coagulation or disseminated intravascular coagulation. The disclosure provides a method of preventing and/or treating a post-pathogenic infection clotting/thrombotic disorder in a patient wherein said thrombotic disease or disorder involves a blood clot thrombus or the potential formation of a blood clot thrombus and further involves stroke and/or one or more transient ischemic attacks (TIA). The disclosure provides a method of preventing and/or treating a post-pathogenic infection clotting/thrombotic disorder in a patient wherein said thrombotic disease or disorder involving a blood clot thrombus or the potential formation of a blood clot thrombus further involves stroke and wherein the subject has non-valvular atrial fibrillation. The disclosure provides a method of preventing and/or treating a post-pathogenic infection clotting/thrombotic disorder in a patient wherein said thrombotic disease or disorder involving a blood clot thrombus or the potential formation of a blood clot thrombus further involves pulmonary hypertension.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION

In order to provide a clear and consistent understanding of the specification and claims, including the scope given to such terms, the following definitions are provided:

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

As used herein, the term “subject” or “patient” encompasses mammals and non-mammals. Examples of mammals include, but are not limited to, any member of the Mammalia class: humans, non-human primates such as chimpanzees, and other apes and monkey species. The subject is preferably mammalian. In some embodiments the subject is a human. The term does not denote a particular age or sex. In other embodiments the subject is an animal, more preferably a non-human mammal. The non-human mammal may be a domestic pet, or animal kept for commercial purposes, e.g., a race horse, or farming livestock such as pigs, sheep or cattle. As such the invention may have veterinary applications. Non-human mammals include rabbits, guinea pigs, rats, mice or other rodents (including any animal in the order Rodentia), cats, dogs, pigs, sheep, goats, cattle (including cows or any animal in the order Bos), horse (including any animal in the order Equidae), donkey, and non-human primates. The subject may be male or female. The subject may be a patient. Examples of non-mammals include, but are not limited to, birds, fish and the like. The patient or subject to be treated may be any animal or human.

The term “administration” of the pharmaceutically active compounds and the pharmaceutical compositions defined herein includes transmucosal application. Nasal, sublingual and buccal administration is particularly preferred in the present invention.

“Ameliorate” or “amelioration” means a lessening of the detrimental effect or severity of the disease in the subject receiving therapy, the severity of the response being determined by means that are well known in the art.

By “compatible” herein is meant that the components of the compositions which comprise the present invention are capable of being commingled without interacting in a manner which would substantially decrease the efficacy of the pharmaceutically active compound under ordinary use conditions.

The terms “effective amount” or “pharmaceutically effective amount” refer to a relatively nontoxic but sufficient amount of the agent to provide the desired biological result. That result can be reduction and/or alleviation of the signs, symptoms, or causes of a disease, such as acute radiation syndrome, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the terms “formulate” refers to the preparation of a pharmaceutical composition in a form suitable for administration to a mammalian patient, preferably a human. Thus, “formulation” can include the addition of pharmaceutically acceptable excipients, diluents, or carriers and pH adjusting agents.

By “pharmaceutically acceptable” or “pharmacologically acceptable” is meant a material which is not biologically or otherwise undesirable, i.e., the material may be administered to an individual without causing undesirable biological effects or interacting in a deleterious manner with any of the components of the composition in which it is contained.

As used herein, a “pharmaceutically acceptable carrier” is a material that is relatively nontoxic and generally inert and does not affect the functionality of the active ingredients adversely. Examples of pharmaceutically acceptable carriers are well known and they are sometimes referred to as diluents, vehicles or excipients. The carriers may be organic or inorganic in nature. In addition, the formulation may contain additives such as flavoring agents, coloring agents, thickening or gelling agents, emulsifiers, wetting agents, buffers, stabilizers, and preservatives such as antioxidants.

The term “pharmaceutical composition” as used herein means a composition that is made under conditions such that it is suitable for administration to, for example, humans, e.g., it is made under GMP conditions and contains pharmaceutically acceptable excipients, e.g., without limitation, stabilizers, pH adjusting agents, bulking agents, buffers, carriers, diluents, vehicles, solubilizers, and binders.

A liquid carrier or vehicle can be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. The prevention of the growth of microorganisms can be accomplished by various antibacterial and antifungal agents, for example, parabens, chlorobutanol, phenol, sorbic acid, thimerosal, and the like.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing or relieving the disease, i.e., causing regression of the disease or its clinical symptoms.

The disclosure provides a method of preventing and/or treating a post-pathogenic infection neurological syndrome in a patient, the method comprising the steps of: selecting a patient in need of preventing and/or treating a post-pathogenic infection neurological syndrome; administering to the patient a nucleoside and/or nucleotide formulation, such as, for example, an oral, enteral, or parenteral nutritional or pharmaceutical formulation; wherein the a post-pathogenic infection neurological syndrome is prevented and/or treated in the patient. The disclosure provides a method wherein the pathogenic infection is selected from the group consisting of viral infection, bacterial infection, fungal infection, parasitic infection, and combinations thereof. The disclosure provides a method, wherein the pathogenic infection is a viral infection. The disclosure provides a method wherein the viral infection is selected from the group consisting of coronavirus, Zika virus, Norovirus, Respiratory Syncytial Virus, Influenza, Adenovirus 5, HPV 11, Lassa Fever virus, Powassan virus, Rift Valley virus, West Nile virus, and combinations thereof. The disclosure provides a method wherein the nucleoside and/or nucleotide oral, enteral, or parenteral nutritional or pharmaceutical formulation is a guanosine formulation.

### Neurological Symptoms

The disclosure provides for the use of a nucleoside and/or nucleotide, such as a guanosine, formulation such as, for example, an oral, enteral, or parenteral nutritional or pharmaceutical formulation, in preventing and treating neurological sequelae of pathogenic infections, such as viral infection. The nucleoside and/or nucleotide formulation, such as a guanosine oral, enteral, or parenteral nutritional or pharmaceutical formulation, is responsible for the preventing and/or treating post-pathogenic infection neurological symptoms.

In addition, the disclosure provides a method of preventing and/or treating postviral neurological syndromes in a patient, the method comprising the steps of: selecting a patient in need of preventing and/or treating postviral neurological syndrome; administering to the patient nucleoside and/or nucleotide oral, enteral, or parenteral nutritional or pharmaceutical formulation, such as a guanosine oral, enteral, or parenteral nutritional or pharmaceutical formulation; wherein the postviral neurological syndrome is prevented and/or treated in the patient. The disclosure provides a method wherein the postviral neurological syndromes is as a result of infection by a virus selected from the group consisting of coronavirus, such as SARS or SARS-Cov2, Zika virus, Norovirus, Respiratory Syncytial Virus, Influenza, Adenovirus 5, HPV 11, Lassa Fever virus, Powassan virus, Rift Valley virus, West Nile virus, and combinations thereof. Araujo, A. Q., Silva, M. T., Araujo, A. P. (2016). Zika virus-associated neurological disorders: a review. Brain 139: 2122-2130. Cardenas, G., Soto-Hernandez, J. L., Diaz-Alba, A., Ugalde, Y., Merida-Puga, J., Rosetti, M., Sciutto, E. (2014). Neurological events related to influenza A (H1N1) pdm09. Influenza Other Respir Viruses 8: 339-346. Chen, S. Y., Tsai, C. N., Lai, M. W., Chen, C. Y., Lin, K. L., Lin, T. Y., Chiu, C. H. (2009). Norovirus infection as a cause of diarrhea-associated benign infantile seizures. Clin Infect Dis 48: 849-855. Money, J. D., Siddharthan, V., Wang, H. (2013). Neurological approaches for investigating West Nile virus disease and its treatment in rodents. Antiviral Res 100: 535-545. Sil, A., Biswas, T., Samanta, M., Konar, M. C., De, A. K., Chaudhuri, J. (2017). Neurological manifestations in children with dengue fever: an Indian perspective. Trop Doct 47: 145-149. Soloman T. (2016). Flavivirus encephalitis and other neurological syndromes (Japanese encephalitis, WNV, Tick borne encephalits, Dengue, Zika virus. International Journal of Infectious Diseases 45S: −24.

The disclosure provides that a nucleoside and/or nucleotide oral, enteral, or parenteral nutritional or pharmaceutical formulation, such as a guanosine oral, enteral, or parenteral nutritional or pharmaceutical formulation, and compositions and methods of the disclosure will prevent, mitigate, and treat neurological disease resulting from infection by pathogenic agent.

Neurological sequelae of pathogenic infection include: Guillain Bane Syndrome, encephalitis, myelitis, encephalomyelitis, meningitis, meningoencephalitis, facial paralysis, confusion, weakness, cognitive dysfunction and other neurological disorders (Araujo et al., 2016). Many of these neurological complications are believed to be due to autoimmune responses to the infection which lead to immunological cells of the human body attacking nerves and supporting cells. It has been discovered that by providing higher doses of nucleic acids that neurological sequelae of pathogenic infection can be prevented, mitigated, and treated.

In addition, other viruses are well known to have neurological sequellae, including West Nile virus (Money et al., 2013), influenza virus (Cardenas et al., 2014, norovirus (Chen et al., 2009), dengue virus (Sil et al., 2017), and other flaviviridae (Soloman et al., 2016). Neurological disorders accompanying and following Covid-19 are well documented (Ahmad I, Rathore F A. 2020. Neurological manifestations and complications of COVID-19: A Literature Review. J Clin Neurosci 77:8-12, Heneka M T, Golenbock D, Latz E, Morgan D, Brown R. 2020. Immediate and long-term consequences of COVID-19 infections for the development of neurological disease. Alzheimer's Res Ther 12:69, Kreye J, Reincke S M, Pruss H. 2020. Do cross-reactive antibodies cause neuropathology in COVID-19? Nat Rev Immunol 20:645-646, Matschke J, Lutgehetmann M, Hagel C, et al., 2020. Neuropathology of patients with COVID-19 in Germany: a post-mortem case series. Lancet Neurology 19:919-929).

### Clotting/Thrombotic Disorders

Thrombotic diseases are the primary indications for thrombin inhibition, because of thrombin's location in the coagulation cascade and, in turn, the importance of the coagulation cascade in the progression of blood clotting processes. However, without wishing to be bound by any theory, it is believed the coagulation cascade in general, and thrombin in particular, is important in a variety other disease states.

This inhibitory action is useful in the treatment of a variety of thrombotic disorders, such as, but not limited to, acute vascular diseases such as acute coronary syndromes; venous-arterial- and cardiogenic thromboembolisms; the prevention of other states such as disseminated intravascular coagulation, or other conditions that involve the presence or the potential formation of a blood clot thrombus.

In some embodiments of the methods, the disease or disorder can be a thrombotic disease or disorder and/or can involves a blood clot thrombus or the potential formation of a blood clot thrombus. In some embodiments, the thrombotic disease or disorder can include acute coronary syndrome, thromboembolism, and/or thrombosis. In some embodiments, the thromboembolism includes venous thromboembolism, arterial thromboembolism, and/or cardiogenic thromboembolism. In some embodiments, the venous thromboembolism includes deep vein thrombosis and/or pulmonary embolism. In some embodiments, the deep vein thrombosis and/or pulmonary embolism can occur following a medical procedure. In some embodiments, the thrombotic disease or disorder can involve dysfunctional coagulation or disseminated intravascular coagulation. In some embodiments, the subject can be undergoing percutaneous coronary intervention (PCI). In some embodiments, the thrombotic disease or disorder can involve a blood clot thrombus or the potential formation of a blood clot thrombus and can further involve stroke and/or one or more transient ischemic attacks (TIA). In some embodiments, the thrombotic disease or disorder involving a blood clot thrombus or the potential formation of a blood clot thrombus can further involve stroke, and the subject can have non-valvular atrial fibrillation.

In some embodiments, the thrombotic disease or disorder can involve a blood clot thrombus or the potential formation of a blood clot thrombus and can further involve pulmonary hypertension. In some embodiments, the pulmonary hypertension can be caused by one or more left heart disorder and/or chronic thromboembolic disease. In some embodiments, the pulmonary hypertension can be associated with one or more lung disease, including pulmonary fibrosis (idiopathic or otherwise), and/or hypoxia.

In some embodiments, the disease or disorder can include fibrosis, Alzheimer's Disease, multiple sclerosis, pain, cancer, inflammation, and/or Type I diabetes mellitus. In some embodiments, the disease or disorder can involve recurrent cardiac events after myocardial infarction.

In some embodiments, the venous thromboembolism can be associated with formation of a thrombus within a vein associated with one or more acquired or inherited risk factors and/or embolism of peripheral veins caused by a detached thrombus. In some embodiments, the one or more risk factors can include a previous venous thromboembolism.

In some embodiments, the cardiogenic thromboembolism can be due to formation of a thrombus in the heart associated with cardiac arrhythmia, heart valve defect, prosthetic heart valves or heart disease, and/or embolism of peripheral arteries caused by a detached thrombus. In some embodiments, the detached thrombus can be in the brain (ischemic stroke). In some embodiments, the detached thrombus can cause a transient ischemic attack (TIA). In some embodiments, the cardiogenic thromboembolism can be due to non-valvular atrial fibrillation. In some embodiments, the thrombosis can be arterial thrombosis. In some embodiments, the arterial thrombosis can be due to one or more underlying atherosclerotic processes in the arteries. In some embodiments, the one or more underlying atherosclerotic processes in the arteries can obstruct or occlude an artery, cause myocardial ischemia (angina pectoris, acute coronary syndrome), cause myocardial infarction, obstruct or occlude a peripheral artery (ischemic peripheral artery disease), and/or obstruct or occlude an artery after a procedure on a blood vessel (reocclusion or restenosis after transluminal coronary angioplasty, reocclusion or restenosis after percutaneous transluminal angioplasty of periphery arteries).

In some embodiments, the treatment or prevention can include an adjunct therapy. In some embodiments, the subject can have myocardial infarction, and said adjunct therapy can be in conjunction with thrombolytic therapy. In some embodiments, the subject can have unstable angina pectoris, thrombosis, and/or heparin-induced thrombocytopenia, and said adjunct therapy can be in combination with antiplatelet therapy. In some embodiments, the subject can have non-valvular atrial fibrillation, and said adjunct therapy can be in conjunction with one or more other therapies. In some embodiments, the subject can have at least one of coronary artery disease and heart failure, and wherein said adjunct therapy can be in combination with antiplatelet therapy. In some embodiments, the subject can further have valvular or non-valvular atrial fibrillation. In some embodiments, the subject can have valvular or non-valvular atrial fibrillation and can be undergoing percutaneous coronary intervention with a stent, and the adjunct therapy can be in combination with antiplatelet therapy.

### Nucleotides

The oral, enteral, or parenteral nutritional or pharmaceutical formulations of the present disclosure may comprise nucleotides and/or nucleotide precursors selected from the group consisting of nucleosides, purine bases, pyrimidine bases, ribose and deoxyribose. The nucleotide may be in monophosphate, diphosphate, or triphosphate form. The nucleotide may be a ribonucleotide or a deoxyribonucleotide. The nucleotides may be monomeric, dimeric, or polymeric (including RNA and DNA). The nucleotide may be present in the nutritional composition as a free acid or in the form of a salt, preferably a monosodium salt. In some embodiments, the oral, enteral, or parenteral nutritional or pharmaceutical formulation as disclosed herein includes nucleotides such that the composition provides a synergistic benefit to the end user, such as a synergistic benefit in modulating anti-viral immune responses and dampening inflammation and/or improving intestinal barrier integrity. The composition of the disclosure may optionally contain other substances that may have a beneficial effect, such as lactoferrin, nucleotides, nucleosides, immunoglobulins, CMP equivalents (cytidine 5′-monophosphate, free acid), UMP equivalents (uridine 5′-monophosphate, disodium salt), AMP equivalents (adenosine 5′-monophosphate, free acid), GMP equivalents (guanosine 5′-monophosphate, disodium salt), and combinations thereof.

Suitable nucleotides and/or nucleosides for use in the oral, enteral, or parenteral nutritional or pharmaceutical formulation as disclosed herein include one or more of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, and inosine 5′-monophosphate.

The nucleotides are present in the oral, enteral, or parenteral nutritional or pharmaceutical formulation as disclosed herein in total amounts of nucleotides, for example guanosine monophosphate, of at least about 5 mg/L, including at least about 10 mg/L, about 25 mg/L, about 50 mg/L, about 100 mg/L, about 150 mg/L, about 200 mg/L, about 250 mg/L, 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L, about 900 mg/L, about 950 mg/L, about 999 mg/L, about 1000 mg/L, including from about 10 mg/L to about 200 mg/L, including from about 42 500 mg/L to about 1000 mg/L, and including at least about 999 mg/L of the nutritional product, such as guanosine, of the oral, enteral, or parenteral nutritional or pharmaceutical formulation. In exemplary embodiments as disclosed herein, the oral, enteral, or parenteral nutritional or pharmaceutical formulation may deliver to a patient about 50 mg/day, about 75 mg/day, about 100 mg/day, about 150 mg/day, about 200 mg/day, about 250 mg/day, about 300 mg/day, about 350 mg/day, about 400 mg/day, about 450 mg/day, about 500 mg/day, about 550 mg/day, about 600 mg/day, about 650 mg/day, about 700 mg/day, about 750 mg/day, about 800 mg/day, about 850 mg/day, about 900 mg/day, about 950 mg/day, about 999 mg/day, about 1000 mg/day, about 1500 mg/day, about 2000 mg/day, about 2500 mg/day, about 3000 mg/day, about 3500 mg/day, about 4000 mg/day of nucleotide, such as guanosine. In exemplary embodiments, the formulation provides about 250 mg/day, about 500 mg/day, about 1000 mg/day, about 2000 mg/day, or about 4000 mg/day of nucleotide, such as guanosine. In exemplary embodiments, the formulation provides about 250 mg/day, about 500 mg/day, about 1000 mg/day, about 2000 mg/day, or about 4000 mg/day of nucleotide, such as guanosine monophosphate.

The human doses of guanosine monophosphate (also known as disodium guanylate) which could be used per day, per person, in 2 or 3 divided doses are: 250 mg, 500 mg, 1000 mg, 2000 mg, and 4000 mg. These levels of guanosine monophosphate were shown in a small clinical trial to be safe (Ekelman, K. and Raffaele, K. C., 1993. Disodium 5′-duanylate and Disodium 5′-lnosinate. Excerpted from: World Health Organization. Toxicological evaluation of certain food additives and contaminants. WHO Food Additives Series, No. 32, 1993. No. 788 on INCHEM. www.inchem.org/documents/jecfa/jecmono/v32je0.6htm cited in www.who.int/ipcs/publications/jecfa/monograpsh/en/

Accessed:6/16/06; citing Mitoma, C, Stone, H., Davis, P. 1972. Effects of feeding purine containing flavor enhancer (ST-1) on Uric acid levels in man. Unpublished report from Stanford Research Institute, Menlo Park, California, 94025, USA. Submitted to WHO by the U.S. Food and Drug Administration.)

### Product Form

The oral, enteral, or parenteral nutritional or pharmaceutical formulation as disclosed herein of the present disclosure may be formulated and administered in any known or otherwise suitable oral, enteral, or parenteral product form. Any solid, liquid, semi-solid, and semi-liquid, or powder product form, including combinations or variations thereof, are suitable for use herein, provided that such forms allow for safe and effective oral delivery to the individual of the essential ingredients as also defined herein.

The oral, enteral, or parenteral nutritional or pharmaceutical formulation as disclosed herein may be in any product form comprising the ingredients described herein, and which is safe and effective for oral, enteral, or parenteral administration. The oral, enteral, or parenteral nutritional or pharmaceutical formulation as disclosed herein may be formulated to include only the ingredients described herein or may be modified with optional ingredients to form a number of different product forms.

The oral, enteral, or parenteral nutritional or pharmaceutical formulation as disclosed herein are desirably formulated as dietary product forms, which are defined herein as those embodiments comprising the ingredients of the present disclosure in a product form that then contains at least one of fat, protein, and carbohydrate, and preferably also contains vitamins, minerals, or combinations thereof.

The oral, enteral, or parenteral nutritional or pharmaceutical formulation as disclosed herein may be formulated with sufficient kinds and amounts of nutrients to provide a sole, primary, or supplemental source of nutrition, or to provide a specialized nutritional product for use in individuals afflicted with specific diseases or conditions or with a targeted nutritional benefit as described below.

### Nutritional Liquids

Nutritional liquids include both concentrated and ready-to-feed nutritional liquids. These nutritional liquids are most typically formulated as suspensions or emulsions, although other liquid forms are within the scope of the present disclosure.

Nutritional emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional emulsions may be and typically are shelf stable. The nutritional emulsions typically contain up to about 95% by weight of water, including from about 50% to about 95%, also including from about 60% to about 90%, and also including from about 70% to about 85%, of water by weight of the nutritional emulsions. The nutritional emulsions may have a variety of product densities, but most typically have a density greater than about 1.03 g/mL, including greater than about 1.04 g/mL, including greater than about 1.055 g/mL, including from about 1.06 g/mL to about 1.12 g/mL, and also including from about 1.085 g/mL to about 1.10 g/mL.

The nutritional emulsions may have a caloric density tailored to the nutritional needs of the ultimate user, although in most instances the emulsions comprise generally at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the emulsion may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter.

The nutritional emulsion may have a pH ranging from about 3.5 to about 8, but are most advantageously in a range of from about 4.5 to about 7.5, including from about 5.5 to about 7.3, including from about 6.2 to about 7.2.

Although the serving size for the nutritional emulsion can vary depending upon a number of variables, a typical serving size is generally at least about 1 mL, or even at least about 2 mL, or even at least about 5 mL, or even at least about 10 mL, or even at least about 25 mL, including ranges from about 1 mL to about 300 mL, including from about 4 mL to about 250 mL, and including from about 10 mL to about 240 mL.

### Nutritional Solids

The nutritional solids may be in any solid form but are typically in the form of flowable or substantially flowable particulate compositions, or at least particulate compositions. Particularly suitable nutritional solid product forms include spray dried, agglomerated and/or dry blended powder compositions. The compositions can easily be scooped and measured with a spoon or similar other device, and can easily be reconstituted by the intended user with a suitable aqueous liquid, typically water, to form a nutritional composition for immediate oral or enteral use. In this context, “immediate” use generally means within about 48 hours, most typically within about 24 hours, preferably right after reconstitution.

The nutritional powders may be reconstituted with water prior to use to a caloric density tailored to the nutritional needs of the ultimate user, although in most instances the powders are reconstituted with water to form compositions comprising at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the reconstituted powder may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter.

### Enteral Formulations

Numerous enteral formulations are utilized in patients with a hyper-metabolic state as effected by burns, trauma, surgery and in patients suffering from malnutrition, chronic illness and in patients suffering from disorders resulting from prolonged periods of reduced oral intake resulting from cerebral vascular accidents, gastrointestinal diseases, or a comatose state. In general, enteral nutrition compositions may be administered orally or by tube feeding. The use of enteral compositions (EN) has provided benefits and advantages as compared to total parenteral nutrition (TPN). The recommendation of the use of enteral compositions is based on recent clinical findings that demonstrate that the use of elemental diets results in fewer complications, reduced patient length of stay in the intensive care unit (ICU), and reduced cost, when compared to TPN.

Elemental diets are composed of low molecular weight nutrients that require minimal digestive and absorptive capability. The protein source consists of free amino acids and in particular essential and nonessential amino acids. The carbohydrate portion of such compositions is typically composed of glucose and hydrolyzed cornstarch (maltodextrin), while the fat content is usually low and primarily consists of essential fatty acids. These diets are characterized by minimal residue in the intestines, because of the efficient absorption of the nutrients provided in an elemental form.

Elemental formulations are, by nature, hyperosmolar (greater than 300 mOsm/kg H₂O, where mOsm=milliosmoles, the osmotic pressure of a solution is the external pressure that must be applied to a solution to prevent the diffusion of solvent from pure solvent into the solution), and can cause diarrhea. Therefore, feeding is initiated using low delivery rates, which has been seen to increase the patient's tolerance.

Different enteral formulations and oral nutritional adjuncts or supplements are available i.e. ISOCAL, OSMOLITE, ENSURE, SUSTACAL, ENSURE PLUS, MAGNACAL, TRAUMACAL, ISOTEIN HN, VIVONEX T.E.N., IMPACT, IMMUN-AID, PEPTAMEN, etc. Generally, pursuant to the present invention, a ready-to-use enteral formulation is provided. The formulation can provide the total nutritional requirements of the intensive care patient or can act as a supplement. The product is designed preferably to be fed to the patient by tube. The product can be provided, for example, in cans or a spike and hang bag. The product is ready to use and does not require reconstitution or mixing prior to use. In an embodiment, the present invention provides a method for providing nutritional support to patients comprising the step of administering a therapeutically effective amount of a composition. The composition preferably includes a protein source; a carbohydrate source; and a lipid source. The protein source is produced with the use of pancreatic enzymes, resulting in a unique peptide profile.

In a preferred embodiment, the enteral formulation has a high caloric content. In an embodiment, preferably, the caloric content is between approximately 1.3 to about 1.5 Kcal/ml. Providing a moderate-to-high caloric intake is necessary to spare protein. Caloric needs in severe trauma, burn, and post-surgical patients typically range from 25 to about 35 Kcal/Kg, e.g., 1800 to 2500 Kcal for a convalescing 70 Kg adult. In fact, severe burn patients can require even higher caloric needs.

Additionally, due to increased metabolic activity, such patients require high protein intake to reduce negative nitrogen balance and support wound repair. Protein needs average 2.0 g of protein per Kg body weight or, e.g., 140 grams of protein per day for a convalescing 70 Kg adult. Therefore, the formulation has a high protein content, preferably at least approximately 22% of the calories of the product are provided as protein. In an embodiment, up to 28% of the calories are provided as protein.

Pursuant to the present invention, the protein source of the composition preferably includes a protein hydrolysate and free amino acids. The use of protein hydrolysate and free amino acids reduces the potential for nutrient malabsorption. A variety of hydrolyzed proteins can be utilized in the present invention. Suitable examples include casein hydrolysate and whey hydrolysate. Preferably, the protein source includes approximately 80% to 85% of protein hydrolysate and approximately 15% to 20% of free amino acids.

In an embodiment, the protein hydrolysate in combination with free amino acids contains less than approximately 20% free amino acids, by weight, and less than approximately 20% peptides, by weight, with a chain length of more than nine amino acids. The hydrolysate of the present invention preferably contains less than approximately 35% peptides, by weight, with a chain-length of more than five amino acids.

As noted above, the protein source of the present invention includes a portion as free amino acids. In an embodiment, the protein source is enriched with arginine and proline as free amino acids. The hydrolysate source also preferably includes a sufficient amount of cysteine to replenish intracellular glutathione in the patient. Providing a high arginine, proline and/or cysteine content promotes wound healing and tissue repair/cell division.

In an embodiment, approximately 25% of the total caloric content of the product is protein. Pursuant to the present invention, either a single hydrolyzed protein or a combination of at least two hydrolyzed proteins can be utilized. For instance, in an embodiment, approximately 80% to 85% of the protein will be partially hydrolyzed casein, approximately 13% to 15% arginine and approximately 4% to 6% proline. In another embodiment, approximately 50% to 55% of the protein will be partially hydrolyzed casein, approximately 30% to 35% partially hydrolyzed whey protein and approximately 13% to 15% will be arginine. In choosing the protein source, the present invention maximizes the natural available levels of desirable amino acid such as arginine, cysteine, proline and glutamine at the highest bioavailability and product stability.

In certain embodiments the oral, enteral, or parenteral nutritional or pharmaceutical formulation as disclosed herein contains may comprise nucleotides and/or nucleotide precursors selected from the group consisting of nucleosides, purine bases, pyrimidine bases, ribose and deoxyribose. The nucleotide may be in monophosphate, diphosphate, or triphosphate form. The nucleotide may be a ribonucleotide or a deoxyribonucleotide. The nucleotides may be monomeric, dimeric, or polymeric (including RNA and DNA). The nucleotide may be present in the nutritional composition as a free acid or in the form of a salt, preferably a monosodium salt. Suitable nucleotides and/or nucleosides for use in the nutritional compositions include one or more of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, and inosine 5′-monophosphate.

The nucleotides are present in the oral, enteral, or parenteral nutritional or pharmaceutical formulation as disclosed herein, for example, in total amounts of nucleotides of at least about 5 mg/L, including at least about 10 mg/L, about 25 mg/L, about 50 mg/L, about 100 mg/L, about 150 mg/L, about 200 mg/L, about 250 mg/L, 300 mg/L, about 350 mg/L, about 400 mg/L, about 450 mg/L, about 500 mg/L, about 550 mg/L, about 600 mg/L, about 650 mg/L, about 700 mg/L, about 750 mg/L, about 800 mg/L, about 850 mg/L, about 900 mg/L, about 950 mg/L, about 999 mg/L, about 1000 mg/L, including from about 10 mg/L to about 200 mg/L, including from about 500 mg/L to about 1000 mg/L, and including at least about 999 mg/L of the nutritional product, such as guanosine.

The oral, enteral, or parenteral nutritional or pharmaceutical formulation as disclosed herein also includes a lipid fraction. Preferably, approximately 33% to about 45% of the formulation, by calories, is provided as a lipid. In a preferred embodiment, 39% of the calories are provided as a lipid.

The lipid fraction contains significant amounts of omega-3 rich fatty acids and medium chain triglycerides (MCTs). Preferably, the lipid fraction comprises approximately 40% to about 60% by calories MCTs. MCTs are more easily absorbed and metabolized as compared to long chain triglycerides (LCTs). The use of MCTs will reduce the risk of the potential for nutrient malabsorption. A low omega-6 content and a high omega-3 content are provided. Preferably, the ratio of omega-6 to omega-3 fatty acids is less than 2.0:1. The low omega-6:omega-3 ratio reduces the incidence and severity of inflammatory reactions. Omega-3 fatty acids may modulate the negative, immune-mediated reactions brought about by high omega-6 intake. Therefore, oil blends which contain omega-3 (or are, at a minimum, low in omega-6) are preferred.

Accordingly, in an embodiment, a fish oil rich in omega-3 fatty acids is preferred, as fish oils contain two longer chain length omega-3 fatty acids: eicosapentaenoic acid (EPA, C22:5, n3) and docosahexaenoic acid (DHA, C22:6, n3). Soy oil is also preferred, in that it contains approximately 7% linolenic acid (C18:3, n3), in order to ensure that a safe minimum level of shorter length omega-3 fatty acids is delivered, and also contains approximately 50-55% linoleic acid (C18:2, n-6), in order to ensure that a safe minimum level of omega-6 fatty acids is delivered (essential fatty acids). In an embodiment of the present invention, the lipid component comprises by weight 50% MCT, 25% fish oil and 25% soy oil (includes soy oil and soy lecithin).

In addition to the ability of omega-3 to modulate inflammatory reactions, likewise, the antioxidant vitamins and minerals also reduce the incidence of severity of inflammatory reactions. By utilizing a formulation having high protein and fat content, protein and energy requirements are met. However, at the same time, pursuant to the present invention, the formulation includes reduced water and carbohydrate content. This reduces the risk of over hydration, hyperglycemia, and carbohydrate intolerance.

Preferably, the formulation is approximately 35% to about 40%, by calories, carbohydrates. By way of example, the carbohydrates can be chosen from maltodextrin, corn starch, sucrose, and corn syrup solids.

In an embodiment, the oral, enteral, or parenteral nutritional or pharmaceutical formulation as disclosed herein includes soluble or insoluble fiber, and/or carob pod powder or extract that is rich in insoluble tannin. In an enteral product, especially one to be provided by tube feeding, this provides anti-diarrhea characteristics. Magnesium can be reduced below U.S. RDA levels (400 mg/day), further limiting the potential for tube-fed induced diarrhea.

Preferably, anti-oxidant vitamins and minerals are increased to above the U.S. RDAs. This will ensure that the patient receives at least 100% of the U.S. RDA as well as insure that any additional micronutrients that are necessary due to the patient's state will be provided. The formulation, in an embodiment, will provide approximately 5-6 mg/1500 Kcal of beta-carotene.

Beta-carotene is a precursor for Vitamin A and has some unique antioxidant properties. The composition according to the present invention is preferably in a dry powder form, but it may also be prepared in any form suitable for administration to an individual in need of treatment. The liquid composition may further comprise amino acids (i.e., glutamine), sugars (i.e., dextrose), salts (i.e., sodium salts), vitamins (i.e., vitamin A), soy or corn oil, triglycerides, safflower oil and any other component suitable for enteral administrations.

Although in principle the present enteral dietary composition may be administered orally, in a preferred embodiment the present composition is fed to the patient through a naso-enteric tube. In this way, the present composition can more easily by-pass the acidity of the gut. Suitable naso-enteric tubes and the use thereof are described in Ziegler, T., Scientific American, November 1995, which is incorporated herein by reference.

### Antioxidants

In some embodiments, the oral, enteral, or parenteral nutritional or pharmaceutical formulation as disclosed herein includes antioxidants such that the composition provides a synergistic benefit to the end user, such as a synergistic benefit in preventing and/or treating a post-pathogenic infection neurological syndrome in a patient.

Any antioxidants suitable for oral administration may be included for use in the nutritional compositions of the present disclosure, including, for example, vitamin A, vitamin E, vitamin C, retinol, tocopherol, and carotenoids, including lutein, beta-carotene, zeaxanthin, and lycopene, and combinations thereof, for example.

In one specific embodiment, the antioxidants for use in the nutritional compositions include carotenoids, and particularly, combinations of the carotenoids lutein, lycopene, zeaxanthin and/or beta-carotene. Nutritional compositions containing these combinations, as selected and defined herein, can be used to prevent and/or treat a post-pathogenic infection neurological syndrome in a patient.

It is generally preferable that the nutritional compositions comprise at least one of lutein, lycopene, zeaxanthin, and beta-carotene to provide a total amount of carotenoid of from about 0.001 microgm/mL to about 10 microgm/mL. More particularly, the nutritional compositions comprise lutein in an amount of from about 0.001 microgm/mL to about 10 microgm/mL, including from about 0.001 microgm/mL to about 5 microgm/mL, including from about 0.001 microgm/mL to about 0.0190 microgm/mL, including from about 0.001 microgm/mL to about 0.0140 microgm/mL, and also including from about 0.044 microgm/mL to about 5 microgm/mL of lutein. It is also generally preferable that the nutritional compositions comprise from about 0.001 microgm/mL to about 10 microgm/mL, including from about 0.001 microgm/mL to about 5 microgm/mL, from about 0.001 microgm/mL to about 0.0130 microgm/mL, including from about 0.001 microgm/mL to about 0.0075 microgm/mL, and also including from about 0.0185 microgm/mL to about 5 microgm/mL of lycopene. It is also generally preferable that the nutritional compositions comprise from about 1 microgm/mL to about 10 microgm/mL, including from about 1 microgm/mL to about 5 microgm/mL, including from about 0.001 microgm/mL to about 0.025 microgm/mL, including from about 0.001 microgm/mL to about 0.011 microgm/mL, and also including from about 0.034 microgm/mL to about 5 microgm/mL of beta-carotene. It should be understood that any combination of these amounts of beta-carotene, lutein, zeaxanthin, and lycopene can be included in the nutritional compositions of the present disclosure. Other carotenoids may optionally be included in the nutritional compositions as described herein. Any one or all of the carotenoids included in the nutritional compositions described herein may be from a natural source, or artificially synthesized. In one particular embodiment, the nutritional composition comprises a combination of 2′FL and lycopene.

### Macronutrients

The nutritional compositions may be formulated to include at least one of protein, fat, and carbohydrate. In many embodiments, the nutritional compositions will include protein, carbohydrate and fat.

Although total concentrations or amounts of the fat, protein, and carbohydrates may vary depending upon the product type (i.e., human milk fortifier, preterm infant formula, infant formula, etc.), product form (i.e., nutritional solid, powder, ready-to-feed liquid, or concentrated liquid) and targeted dietary needs of the intended user, such concentrations or amounts most typically fall within one of the following embodied ranges, inclusive of any other essential fat, protein, and/or carbohydrate ingredients as described herein.

For the compositions and methods as disclosed herein, carbohydrate concentrations most typically range from about 5% to about 40%, including from about 7% to about 30%, including from about 10% to about 25%, by weight of the formula; fat concentrations most typically range from about 1% to about 30%, including from about 2% to about 15%, and also including from about 3% to about 10%, by weight of the formula; and protein concentrations most typically range from about 0.5% to about 30%, including from about 1% to about 15%, and also including from about 2% to about 10%, by weight of the formula.

### Pathogenic Infections

In a preferred embodiment the pathogen is an intracellular pathogen, i.e., a pathogen capable of growing and reproducing inside the cells of a host. Bacterial examples which may be prevented and/or treated by the compositions and methods of the invention include but are not limited to Francisella tularensis, Listeria monocytogenes, Salmonella, Brucella, Legionella, Mycobacterium, Nocardia, Rhodococcus equi, Yersinia, Neisseria meningitidis, Chlamydia, Rickettsia, Coxiella, Mycobacterium, such as Mycobacterium leprae and Treponema pallidum. Fungal examples include but are not limited to Histoplasma capsulatum, Cryptococcus neoformans and Pneumocystis jirovecii. Examples of protozoa include but are not limited to Apicomplexans (e.g. Plasmodium spp., Toxoplasma gondii and Cryptosporidium parvum) and Trypanosomatids (e.g. Leishmania spp. and Trypanosoma cruzi).

The following is an exemplary but non-limiting discussion of various disease agents that could be the subject of the prevention and/or treatment of neurological symptoms in accordance with the present invention.

### Bacterial Pathogens

There are hundreds of bacterial pathogens in both the Gram-positive and Gram-negative families that cause significant illness and mortality around the word, despite decades of effort developing antibiotic agents. Antibiotic resistance is a growing problem in bacterial disease. Bacterial pathogens may be prevented and/or treated by the compostions and methods of the invention.

One of the bacterial diseases with highest disease burden is tuberculosis, caused by the bacterium Mycobacterium tuberculosis, which kills about 2 million people a year, mostly in sub-Saharan Africa. Pathogenic bacteria contribute to other globally important diseases, such as pneumonia, which can be caused by bacteria such as Streptococcus and Pseudomonas, and food borne illnesses, which can be caused by bacteria such as Shigella, Campylobacter, and Salmonella. Pathogenic bacteria also cause infections such as tetanus, typhoid fever, diphtheria, syphilis, and leprosy.

Conditionally pathogenic bacteria are only pathogenic under certain conditions, such as a wound facilitates entry of bacteria into the blood, or a decrease in immune function. For example, Staphylococcus or Streptococcus are also part of the normal human flora and usually exist on the skin or in the nose without causing disease, but can potentially cause skin infections, pneumonia, meningitis, and even overwhelming sepsis, a systemic inflammatory response producing shock, massive vasodilation and death. Some species of bacteria, such as Pseudomonas aeruginosa, Burkholderia cenocepacia, and Mycobacterium avium, are opportunistic pathogens and cause disease mainly in people suffering from immunosuppression or cystic fibrosis.

Other bacteria invariably cause disease in humans, such as obligate intracellular parasites (e.g., Chlamydophila, Ehrlichia, Rickettsia) that are capable of growing and reproducing only within the cells of other organisms. Still, infections with intracellular bacteria may be asymptomatic, such as during the incubation period. An example of intracellular bacteria is Rickettsia. One species of Rickettsia causes typhus, while another causes Rocky Mountain spotted fever. Chlamydia, another phylum of obligate intracellular parasites, contains species that can cause pneumonia or urinary tract infection and may be involved in coronary heart disease. Mycobacterium, Brucella, Francisella, Legionella, and Listeria can exist intracellular, though they are facultative (not obligate) intracellular parasites.

Gram-positive bacteria include Staphylococcus aureus; Staphylococcus epidermidis; Staphylococcus saprophyticus; Streptococcus pyogenes (Lancefield group A, beta-hemolytic); Streptococcus agalactiae (Lancefield group B, beta-hemolytic); Streptococcus Viridans group (most are alpha-hemolytic) including, for example, the Mitus group (S. mitus, S. sanguis, S. parasanguis, S. gordonii, S. crista, S. infantis, S. oralis, S. peroris), the Salivarius group (S. salivarius, S. vestibularis, S. thermophilus), the Mutans group (S. mutans, S. sobrinus, S. criceti, S. rattus, S. downei, S. macacae), and the Anginosus group (S. anginosus, S. constellatus, S. intermedius); Streptococcus, e.g., S. bovis, S. equinus (Lancefield group D, alpha-hemolytic); Streptococcuspneumoniae (no Lancefield antigen; alpha-hemolytic); Peptostreptococcus and Peptococcus; Entercoccus faecalis; Enterococcus faeccium; Cornybacterium diphtheria; Bacillus anthracis; Bacillus cereus; Clostridium C. botulinum (more rarely, C. baratii and C. butyricum); Clostridium tetani; Clostridium perfringens; Clostridium difficile; Clostridium sordellii; Listeria monocytogenes; Actinomyces israelii; Nocardia asteroids; Streptomyces.

Gram-negative bacteria include Neisseria meningitides; Neisseria gonorrhoeae; Moraxella (subgenera Branhamella) catarrhalis; Kingella (most commonly kingae); Acinetobacter baumannii, Oligella ureolytica; Oligella urethralis; Escherichia coli; Shigella (S. dysenteriae, S. flexneri, S. boydii, S. sonnei); Salmonella non typhoidal, including S. enterica serotype enteritidis, S. enterica serotype typhimurium, S. enterica serotype Choleraesuis, S. bongori, Salmonella S. enterica serotype Typhi; Yersinia enterocolitica, Klebsiella pneumoniae; Proteus mirabilis; Enterobacter; Cronobacter (formerly called Enterobacter sakazakii); Serratia; Edwardsiella; Citrobacter; Hafnia; Providencia; Vibrio cholera; Vibrio parahemolyticus; Campylobacter; Helicobacter (formerly called Campylobacter) pylori, Pseudomonas aeruginosa; Burkholderia cepacia; Burkholderia mallei; Burkholderia pseudomallei; Stenotrophomonas maltophilia; Bacteroides fragilis, Bacteroides melaninogenicus; Fusobacterium; Haemophilus influenza; Haemophilus ducreyi; Gardnerella (formerly called Haemophilus) vaginalis; Bordetella pertussis; Legionella; Yersinia pestis; Francisella tularensis; Brucella B. melitensis (infects sheep/goats); B. abortus (abortions in cows); B. suis (pigs); B. canis (dogs); B. maris (marine animals); Pasteurella multocida; Streptobacillus moniliformis; Spirillum minus; Treponema pallidum; Treponema pallidum subspecies pertenue; Treponema pallidum subspecies endemicum; Treponema pallidum subspecies carateum; Borrelia burgdorferi; Borrelia; Leptospira; Chlamydia trachomatis; Chlamydia pneumonia; Chlamydia psittaci; Rickettsiae rickettsia; Rickettsiae akari; Rickettsiae prowazekii; Rickettsiae typhi; Rickettsiae tsutsugamushi; Rickettsiae parkeri; Rickettsiae africae; Rickettsia conorii; Rickettsia australis; Rickettsia siberica; Rickettsia japonica; Bartonella Quintana; Bartonella henselae; Bartonella bacilliformis; Coxiella burnetii; Ehrlichia; Anaplasma phagocytophilum; Neorickettsia; Orientia; Klebsiella granulomatis (formerly called Calymmatobacterium granulomatis); Capnocytophaga.

Other bacteria include Mycobacterium tuberculosis; Mycobacterium bovis; Mycobacterium leprae; Mycobacterium avium-intracellulare or avium complex (MAI or MAC); Mycobacterium ulcerans; Mycobacterium kansasii; Mycobacterium marinum; Mycobacterium scrofulaceum; Mycobacterium fortuitum; Mycobacterium chelonei; Mycobacterium abscessus; Mycoplasma pneumonia; Ureaplasma urealyticum.

### Viral Pathogens

Viruses include DNA and RNA viruses. These include respiratory viruses such as Adenoviruses, Avian influenza, Influenza virus type A, Influenza virus type B, Measles, Parainfluenza virus, Respiratory syncytial virus (RSV), Rhinoviruses, and SARS coronavirus, gastro-enteric viruses such as Coxsackie viruses, enteroviruses such as Poliovirus and Rotavirus, hepatitis viruses such as Hepatitis B virus, Hepatitis C virus, Bovine viral diarrhea virus (surrogate), herpes viruses such as Herpes simplex 1, Herpes simplex 2, Human cytomegalovirus, and Varicella zoster virus, retroviruses such as Human immunodeficiency virus 1 (HIV-1), and Human immunodeficiency virus 2 (HW-2), as well as Dengue virus, Hantavirus, Hemorrhagic fever viruses, Lymphocytic choriomeningitis virus, Smallpox virus, Ebola virus, Rabies virus, West Nile virus (WNV) and Yellow fever virus.

Examples of viruses whose neurologic sequalae which may be prevented and/or treated by the compositions and methods of the disclosure include Parvoviridae; Papovaviridae (Human papilloma virus (HPV); BK polyomavirus; JC polyomavirus); Adenoviridae (Adenovirus, types 40 and 41); Herpesviridae (simplex virus type 1 (HHV-1); Herpes simplex virus type 2 (HHV-2); Macacine herpesvirus 1; Varicella-zoster virus (VZV; HHV-3); Epstein-Barr virus (EBV; HHV-4); Cytomegalovirus (CMV; HHV-5); Human Herpesvirus 6 (HHV-6); HHV-7; Kaposi's sarcoma-associated herpesvirus (HHV-8); Hepadnaviridae (Hepatitis B virus); Poxviridae (Smallpox (Variola major); Alastrim (Variola minor); Vaccinia; Cowpox; Monkeypox; Goat pox, pseudocowpox virus, bovine papular stomatitis virus, tanapox, volepox and related pox viruses such as avipox, buffalopox, racoonpox, squirrelpox, etc.); Molluscum contagiosum; Picornaviridae (Polio virus; Coxsackie A virus; Coxsackie B; virus; Foot and mouth disease; ECHO virus; Hepatitis A virus; Rhinovirus); Astroviridae; Caliciviridae (Norwalk virus; Norovirus; Sapoviruses; Hepatitis E virus); Reoviridae (Rotavirus); Togaviridae (Alpha viruses; Western equine encephalitis (WEE) virus; Eastern equine encephalitis (EEE) virus; Venezuelan equine encephalitis (VEE) virus; Chikungunya virus; Rubivirus (rubella)); Flaviviridae (Yellow fever virus; Dengue virus; St. Louis encephalitis virus; Japanese encephalitis virus; Tick-borne encephalitis virus; Omsk hemorrhagic fever virus; Al Khumra virus; Kyasanur Forest disease virus; Louping ill virus; West Nile virus; Kunjin virus; Murray Valley fever virus; Powassan virus; Hepatitis C virus; Hepatitis G virus); Coronoviridae (Respiratory illness (cold); Severe Acute Respiratory Syndrom)-corona virus (SARS-CoV)); Bunyaviridae (California encephalitis virus; La Crosse virus; Rift Valley fever virus; West Nile virus, Phleboviruses; Sandfly fever virus; Nairovirus; Hantavirus); Orthomyxoviridae (Influenza virus (types A, B & C); Paramyxoviridae (Parainfluenza virus; Respiratory syncytial virus (RSV); Hendra virus disease (formerly equine morbillivirus); Nipah virus encephalitis; Mumps Measles; Newcastle disease virus); Rhabdoviridae (Rabies virus); Filoviridae (Marburg virus (acute hemorrhagic fever); Ebola virus (acute hemorrhagic fever)); Arenaviridae (Lymphocytic choriomeningitis virus; Lassa fever virus; Lujo virus; Chapare virus; Junin virus; Machupo virus; Guanarito virus; Sabia virus); Retroviridae (Human Immunodeficiency virus (HIV) types I and II; Human T-cell leukemia virus (HLTV) type I; Human T-cell leukemia virus (HLTV) type II; Spumaviruses; Xenotropic murine leukemia virus-related (XMRV).

A non-exhaustive list of viruses and their species which can be prevented and/or treated by the compositions and methods of the invention include, for example: Abadina virus (Reoviridae), Abelson murine leukemia virus (Retroviridae), Abras virus (Bunyaviridae), Absettarov virus (Flaviviridae), Abu Hammad virus (Bunyaviridae), Abu Mina virus (Bunyaviridae), Acado virus (Reoviridae), Acara virus (Bunyaviridae), Acciptrid herpesvirus (Herpesviridae), Acheta domestica densovirus (Parvoviridae), Acrobasis zelleri entomopoxvirus (Poxviridae), Adelaide River virus (Rhabdoviridae), Adeno-associated virus (Parvoviridae), Aedes aegypti densovirus (Parvoviridae), Aedes aegypti entomopoxvirus (Poxviridae), Aedes albopictus densovirus (Parvoviridae), Aedes pseudoscutellaris densovirus (Parvoviridae), African green monkey cytomegalovirus (Herpesviridae), African green monkey HHV-like virus (Herpesviridae), African green monkey polyomavirus (Papovaviridae), African horse sickness viruses (Reoviridae), African swine fever virus, African swine fever-like viruses, AG-virus (Bunyaviridae), AG-virus, (Bunyaviridae), Agaricus bisporus virus, Aguacate virus (Bunyaviridae), Ahlum water-borne virus (Tombusviridae), Aino virus (Bunyaviridae), Akabane virus (Bunyaviridae), AKR (endogenous) murine leukemia virus (Retroviridae), Alajuela virus (Bunyaviridae), Alcelaphine herpesvirus (Herpesviridae), Alenquer virus (Bunyaviridae), Aleutian disease virus (Parvoviridae), Aleutian mink disease virus (Parvoviridae), Alfuy virus (Flaviviridae), Allerton virus (Herpesviridae), Allitrich herpesvirus (Herpesviridae), Allomyces arbuscula virus, Almeirim virus (Reoviridae), Almpiwar virus (Rhabdoviridae), Altamira virus, (Reoviridae), Amapari virus (Arenaviridae), American ground squirrel herpesvirus, (Herpesviridae), Amsacta moorei entomopoxvirus (Poxviridae), Amyelosis chronic stunt virus (Caliciviridae), Ananindeua virus (Bunyaviridae), Anatid herpesvirus (Herpesviridae), Andasibe virus (Reoviridae), Anhanga virus (Bunyaviridae), Anhembi virus (Bunyaviridae), Anomala cuprea entomopoxvirus (Poxviridae), Anopheles A virus (Bunyaviridae), Anopheles virus (Bunyaviridae), Antequera virus (Bunyaviridae), Aotine herpesvirus (Herpesviridae), Apeu virus (Bunyaviridae), Aphodius tasmaniae entomopoxvirus (Poxviridae), Apoi virus (Flaviviridae), Aransas Bay virus (Bunyaviridae), Arbia virus (Bunyaviridae), Arboledas virus (Bunyaviridae), Arbroath virus (Reoviridae), Argentine turtle herpesvirus (Herpesviridae), Arkonam virus (Reoviridae), Aroa virus (Flaviviridae), Arphia conspersa entomopoxvirus (Poxviridae), Aruac virus (Rhabdoviridae), Arumowot virus (Bunyaviridae), Asinine herpesvirus (Herpesviridae), Atlantic cod ulcus syndrome virus (Rhabodoviridae), Atlantic salmon reovirus Australia (Reoviridae), Atlantic salmon reovirus Canada (Reoviridae), Atlantic salmon reovirus USA (Reoviridae), Atropa belladorma virus (Rhabdoviridae), Aucuba bacilliform virus, Badnavirus, Aujeszky's disease virus (Herpesviridae), Aura virus (Togaviridae), Auzduk disease virus (Poxviridae), Avalon virus (Bunyaviridae), Avian adeno-associated virus (Parvoviridae), Avian carcinoma, Mill Hill virus (Retroviridae), Avian encephalomyelitis virus (Picornaviridae), Avian infectious bronchitis virus (Coronaviridae), Avian leukosis virus-RSA (Retroviridae), Avian myeloblastosis virus (Retroviridae), Avian myelocytomatosis virus (Retroviridae), Avian nephrites virus (Picornaviridae), Avian paramyxovirus (Paramyxoviridae), Avian reovirus (Reoviridae), B virus (Parvoviridae), B-lymphotropic papovavirus (Papovaviridae), Babahoya virus (Bunyaviridae), Babanki virus (Togaviridae), Baboon herpesvirus (Herpesviridae), Baboon polyomavirus (Papovaviridae), Bagaza virus (Flaviviridae), Bahia Grande virus (Rhabdoviridae), Bahig virus (Bunyaviridae), Bakau virus (Bunyaviridae), Baku virus (Reoviridae), Bald eagle herpesvirus (Herpesviridae), Bandia virus (Bunyaviridae), Bangoran virus (Rhabdoviridae), Bangui virus (Bunyaviridae), Banzi virus (Flaviviridae), Barmah Forest virus (Togaviridae), Barranqueras virus (Bunyaviridae), Barur virus (Rhabdoviridae), Batai virus (Bunyaviridae), Batarna virus (Bunyaviridae), Batken virus (Bunyaviridae), Bauline virus (Reoviridae), Beak and feather disease virus (Circoviridae), BeAn virus (Rhabdoviridae), BeAr virus (Bunyaviridae), Bebaru virus (Togaviridae), Belem virus (Bunyaviridae), Belmont virus ((Bunyaviridae)), Belterra virus (Bunyaviridae), Benevides virus (Bunyaviridae), Benfica virus (Bunyaviridae), Berne virus, (Coronaviridae), Berrimah virus (Rhabdoviridae), Bertioga virus (Bunyaviridae), Bhanj a virus (Bunyaviridae), Bimbo virus (Rhabdoviridae), Bimiti virus (Bunyaviridae), Birao virus (Bunyaviridae), BivensArm virus (Rhabdoviridae), BK virus (Papovaviridae), Bluetongue viruses (Reoviridae), Bobaya virus (Bunyaviridae), Bobia virus (Bunyaviridae), Bobwhite quail herpesvirus (Herpesviridae), Boid herpesvirus (Herpesviridae), Bombyx mori densovirus (Parvoviridae), Boolarra virus (Nodaviridae), Boraceia virus (Bunyaviridae), Border disease virus (Flaviviridae), Boma disease virus, Botambi virus (Bunyaviridae), Boteke virus, (Rhabdoviridae), Bouboui virus (Flaviviridae), Bovine adeno-associated virus (Parvoviridae), Bovine adenoviruses (Adenoviridae), Bovine astrovirus (Astroviridae), Bovine coronavirus (Coronaviridae), Bovine diarrhea virus (Flaviviridae), Bovine encephalitis herpesvirus (Herpesviridae), Bovine enteric calicivirus (Caliciviridae), Bovine enterovirus (Picornaviridae), Bovine ephemeral fever virus (Rhabdoviridae), Bovine herpesvirus (Herpesviridae), Bovine immunodeficiency virus (Retroviridae), Bovine leukemia virus (Retroviridae), Bovine mamillitis virus (Herpesviridae), Bovine papillomavirus (Papovaviridae), Bovine papular stomatitis virus (Poxviridae), Bovine parainfluenza virus (Paramyxoviridae), Bovine parvovirus (Parvoviridae), Bovine polyomavirus (Papovaviridae), Bovine Respiratory Syncytial Virus (Paramyxoviridae), Bovine rhinovirus (Picornaviridae), Bovine syncytial virus (Retroviridae), Bozo virus (Bunyaviridae), Broadhaven virus (Reoviridae), Bruconha virus (Bunyaviridae), Brus Laguna virus (Bunyaviridae), Budgerigar fledgling disease virus (Papovaviridae), Buenaventura virus (Bunyaviridae), Buffalopox virus (Poxviridae), Buggy Creek virus (Togaviridae), Bujaru virus (Bunyaviridae), Bukalasa bat virus (Flaviviridae), Bunyamwera virus (Bunyaviridae), Bunyip creek virus (Reoviridae), Bushbush virus (Bunyaviridae), Bussuquara virus (Flaviviridae), Bwamba virus (Bunyaviridae), Cache Valley virus (Bunyaviridae), Cacipacore virus (Flaviviridae), Caddo Canyon virus (Bunyaviridae), Caimito virus (Bunyaviridae), Calchaqui virus (Rhabdoviridae), California encephalitis virus (Bunyaviridae), California harbor sealpox virus (Poxviridae), Callistephus chinensis chlorosis virus (Rhabdoviridae), Callitrichine herpesvirus (Herpesviridae), Camel contagious ecthyma virus (Poxviridae), Camelpox virus (Poxviridae), Camptochironomus tentans entomopoxvirus (Poxviridae), Cananeia virus (Bunyaviridae), Canarypox virus (Poxviridae), Candiru virus (Bunyaviridae), Canid herpesvirus (Herpesviridae), Caninde virus (Reoviridae), Canine adeno-associated virus (Parvoviridae), Canine adenovirus (Adenoviridae), Canine calicivirus (Caliciviridae), Canine coronavirus (Coronaviridae), Canine distemper virus (Paramyxoviridae), Canine herpesvirus (Herpesviridae), Canine minute virus (Parvoviridae), Canine oral papillomavirus (Papovaviridae), Canine parvovirus (Parvoviridae), Canna yellow mottle virus (Badnavirus), Cape Wrath virus (Reoviridae), Capim virus (Bunyaviridae), Caprine adenovirus (Adenoviridae), Caprine arthritis encephalitis virus (Retroviridae), Caprine herpesvirus (Herpesviridae), Capuchin herpesvirus AL-(Herpesviridae), Capuchin herpesvirus AP-(Herpesviridae), Carajas virus (Rhabdoviridae), Caraparu virus (Bunyaviridae), Carey Island virus (Flaviviridae), Casphalia extranea densovirus (Parvoviridae), Catu virus (Bunyaviridae), Caviid herpesvirus ((Herpesviridae)), CbaAr virus (Bunyaviridae), Cebine herpesvirus (Herpesviridae), Cercopithecine herpesvirus (Herpesviridae), Cervid herpesvirus (Herpesviridae), CG-virus (Bunyaviridae), Chaco virus (Rhabdoviridae), Chagres virus (Bunyaviridae), Chamois contagious ecthyma virus (Poxviridae), Chandipura virus (Rhabdoviridae), Changuinola virus (Reoviridae), Charleville virus (Rhabdoviridae), Chelonid herpesvirus (Herpesviridae), Chelonid herpesvirus (Herpesvirzdae), Chelonid herpesvirus (Herpesviridae), Chenuda virus (Reoviridae), Chick syncytial virus (Retroviridae), Chicken anemia virus (Circoviridae), Chicken parvovirus (Parvoviridae), Chikungunya virus (Togaviridae), Chilibre virus (Bunyaviridae), Chim virus (Bunyaviridae), Chimpanzee herpesvirus (Herpesviridae), Chironomus attenuatus entomopoxvirus (Poxviridae), Chironomus luridus entomopoxvirus (Poxviridae), Chironomus plumosus erltomopoxvirus (Poxviridae), Chobar Gorge virus (Reoviridae), Choristoneura biennis entomopoxvirus (Poxviridae), Choristoneura conflicta entomopoxvirus (Poxviridae), Choristoneura diversuma entomopoxvirus (Poxviridae), Chorizagrotis auxiliars entomopoxvirus (Poxviridae), Chub reovirus Germany (Reoviridae), Ciconiid herpesvirus (Herpesviridae), Clo Mor virus (Bunyaviridae), CoAr-virus (Bunyaviridae), Coastal Plains virus (Rhabdoviridae), Cocal virus (Rhabdoviridae), Coital exanthema virus (Herpesviridae), ColAn-virus (Bunyaviridae), Colocasia bobone disease virus, (Rhabdoviridae), Colorado tick fever virus, (Reoviridae), Columbia SK virus, (Picornaviridae), Columbid herpesvirus, (Herpesviridae), Connecticut virus, (Rhabdoviridae), Contagious ecthyma virus, (Poxviridae), Contagious pustular dermatitis virus, (Poxviridae), Corfu virus, (Bunyaviridae), Corriparta virus, (Reoviridae), Cotia virus, (Poxviridae), Cowpox virus, (Poxviridae), Crimean-Congo hemorrhagic fever virus, (Bunyaviridae), CSIRO village virus, (Reoviridae), Cynara virus, (Rhabdoviridae), Cyprinid herpesvirus, (Herpesviridae), Dabakala virus, (Bunyaviridae), D'Aguilar virus, (Reoviridae), Dakar bat virus, (Flaviviridae), DakArk virus, (Rhabdoviridae), Deer papillomavirus, (Papovaviridae), Demodema boranensis entomopoxvirus, (Poxviridae), Dengue virus, (Flaviviridae), Dengue virus group, (Flaviviridae), Dependovirus, (Parvoviridae), Dera Ghazi Khan virus, (Bunyaviridae), Dera Ghazi Khan virus Group, (Bunyaviridae), Dermolepida albohirtum entomopoxvirus, (Poxviridae), Dhori virus, (Orthomyxoviridae), Diatraea saccharalis densovirus, (Parvoviridae), Dobrava-Belgrade virus, (Bunyaviridae), Dolphin distemper virus, (Paramyxoviridae), Dolphinpox virus, (Poxviridae), Douglas virus, (Bunyaviridae), Drosophila C virus, (Picornaviridae), Dry Tortugas virus, (Bunyaviridae), duck adenovirus, (Adenoviridae), Duck adenovirus, (Adenoviridae), Duck astrovirus, (Astroviridae), Duck hepatitis B virus, (Hepadnaviridae), Duck plague herpesvirus syn. anatid herpesvirus, (Herpesviridae), Dugbe virus, (Bunyaviridae), Duvenhage virus, (Rhabdoviridae), Eastern equine encephalitis virus, (Togaviridae), Ebola virus Filoviridae, Echinochloa hoj a blanca virus; Genus Tenuivirus, Echinochloa ragged stunt virus, (Reoviridae), ectromelia virus, (Poxviridae), Edge Hill virus, (Flaviviridae), Egtved virus syn. viral hemorrhagic septicemia virus, (Rhabdoviridae), Elapid herpesvirus, (Herpesviridae), Elephant loxondontal herpesvirus, (Herpesviridae), Elephant papillomavirus, (Papovaviridae), Elephantid herpesvirus, (Herpesviridae), Ellidaey virus, (Reoviridae), Embu virus, (Poxviridae), Encephalomyocarditis virus, (Picornaviridae), Enseada virus, (Bunyaviridae), Entamoeba virus, (Rhabdoviridae), Entebbe bat virus, (Flaviviridae), Epizootic hemorrhagic disease viruses, (Reoviridae), Epstein-Barr virus, (Herpesviridae), Equid herpesvirus, (Herpesviridae), Equid herpesvirus, (Nerpesviridae), Equid herpesvirus, (Herpesviridae), Equine abortion herpesvirus, (Herpesviridae), Equine adeno-associated virus, (Parvoviridae), Equine adenovirus, (Adenoviridae), Equine arteritis virus, (Arterivirus), Equine cytomegalovirus, (Herpesviridae), Equine encephalosis viruses, (Reoviridae), Equine herpesvirus, (Herpesviridae), Equine infectious anemia virus, (Retroviridae), Equine papillomavirus, (Papovaviridae), Equine rhinopneumonitis virus, (Herpesviridae), Equine rhinovirus, (Picornaviridae), Eret-virus, (Bunyaviridae), Erinaceid herpesvirus, (Herpesviridae), Erve virus, (Bunyaviridae), Erysimum latent virus, Tymovirus, Esocid herpesvirus, (Herpesviridae), Essaouira virus, (Reoviridae), Estero Real virus, (Bunyaviridae), Eubenangee virus, (Reoviridae), Euonymus fasciation virus, (Rhabdoviridae), European bat virus, (Rhabdoviridae), European brown hare syndrome virus, (Caliciviridae), European elk papillomavirus, (Papovaviridae), European ground squirrel cytomegalovirus, (Herpesviridae), European hedgehog herpesvirus, (Herpesviridae), Everglades virus, (Togaviridae), Eyach virus, (Reoviridae), Facey's Paddock virus, (Bunyaviridae), Falcon inclusion body disease, (Herpesviridae), Falconid herpesvirus, (Herpesviridae), Farallon virus, (Bunyaviridae), Felid herpesvirus, (Herpesviridae), Feline calicivirus, (Caliciviridae), Feline herpesvirus, (Herpesviridae), Feline immunodeficiency virus, (Retroviridae), Feline infectious peritonitis virus, (Coronaviridae), Feline leukemia virus, (Retroviridae), Feline parlleukopenia virus, (Parvoviridae), Feline parvovirus, (Parvoviridae), Feline syncytial virus, (Retroviridae), Feline viral rhinotracheitis virus, (Herpesviridae), Fetal rhesus kidney virus, (Papovaviridae), Field mouse herpesvirus, (Herpesviridae), Figulus subleavis entomopoxvirus, (Poxviridae), Fiji disease virus, (Reoviridae), Fin V-virus, (Bunyaviridae), Finkel-Biskis-Jinkins murine sarcoma virus, (Retroviridae), Flanders virus, (Rhabdoviridae), Flexal virus, (Arenaviridae), Flock house virus, Nodaviridae, Foot-and-mouth disease virus A, (Picornaviridae), Foot-and-mouth disease virus ASIA, (Picornaviridae), Foot-and-mouth disease virus, (Picornaviridae), Forecariah virus, (Bunyaviridae), Fort Morgan virus, (Togaviridae), Fort Sherman virus, (Bunyaviridae), Foula virus, (Reoviridae), Fowl adenoviruses, (Adenoviridae), Fowl calicivirus, (Caliciviridae), Fowlpox virus, (Poxviridae), Fraser Point virus, (Bunyaviridae), Friend murine leukemia virus, (Retroviridae), Frijoles virus, (Bunyaviridae), Frog herpesvirus, (Herpesviridae), Fromede virus, (Reoviridae), Fujinami sarcoma virus, (Retroviridae), Fukuoka virus, (Rhabdoviridae), Gabek Forest virus, (Bunyaviridae), Gadget's Gully virus, (Flaviviridae), Galleria mellonella densovirus, (Parvoviridae), Gallid herpesvirus, (Herpesviridae), Gamboa virus, (Bunyaviridae), Gan Gan virus, (Bunyaviridae), Garba virus, (Rhabdoviridae), Gardner-Arnstein feline sarcoma virus, (Retroviridae), Geochelone carbonaria herpesvirus, (Herpesviridae), Geochelone chilensis herpesvirus, (Herpesviridae), Geotrupes sylvaticus entomopoxvirus, (Poxviridae), Gerbera symptomless virus, (Rhabdoviridae), Germiston virus, (Bunyaviridae), Getah virus, (Togaviridae), Gibbon ape leukemia virus, (Retroviridae), Ginger chlorotic fleckvirus, Sobemovirus, Glycine mottle virus, Tombusviridae, Goat herpesvirus, (Herpesviridae), Goatpox virus, (Poxviridae), Goeldichironomus holoprasimus entomopoxvirus, (Poxviridae), Golden shiner reovirus, (Reoviridae), Gomoka virus, (Reoviridae), Gomphrena virus, (Rhabdoviridae), Gonometa virus, (Picornaviridae), Goose adenoviruses, (Adenoviridae), Goose parvovirus, (Parvoviridae), Gordil virus, (Bunyaviridae), Gorilla herpesvirus, (Herpesviridae), Gossas virus, (Rhabdoviridae), Grand Arbaud virus, (Bunyaviridae), Gray Lodge virus, (Rhabdoviridae), Gray patch disease agent of green sea turtle, (Herpesviridae), Great Island virus, (Reoviridae), Great Saltee Island virus, (Reoviridae), Great Saltee virus, (Bunyaviridae), Green iguana herpesvirus, (Herpesviridae), Green lizard herpesvirus, (Herpesviridae), Grey kangaroopox virus, (Poxviridae), Grimsey virus, (Reoviridae), Ground squirrel hepatitis B virus, (Hepadnaviridae), GroupA-K rotaviruses, (Reoviridae), Gruid herpesvirus, (Herpesviridae), GUU-virus, (Bunyaviridae), Guajara virus, (Bunyaviridae), Guama virus, (Bunyaviridae), Guanarito virus, (Arenaviridae), Guaratuba virus, (Bunyaviridae), Guaroa virus, (Bunyaviridae), Guinea pig cytomegalovirus, (Herpesviridae), Guinea pig herpesvirus, (Herpesviridae), Guinea pig type C oncovirus, (Retroviridae), Gumbo Limbo virus, (Bunyaviridae), Gurupi virus, (Reoviridae), H-virus, (Parvoviridae), H virus, (Bunyaviridae), Hamster herpesvirus, (Herpesviridae), Hamster polyomavirus, (Papovaviridae), Hantaan virus, (Bunyaviridae), Hanzalova virus, (Flaviviridae), Hardy-Zuckerman feline sarcoma virus, (Retroviridae), Hare fibroma virus, (Poxviridae), Hart Park virus, (Rhabdoviridae), Hartebeest herpesvirus, (Herpesviridae), Harvey murine sarcoma virus, (Retroviridae), Hazara virus, (Bunyaviridae), HB virus, (Parvoviridae), Hepatitis virus, (Picornaviridae), Hepatitis virus, (Hepadnaviridae), Hepatitis virus, (Flaviviridae), Herpesvirus M, (Herpesviridae), Herpesvirus papio, (Herpesviridae), Herpesvirus platyrrhinae type, (Herpesviridae), Herpesvirus pottos, (Herpesviridae), Herpesvirus saimiri, (Herpesviridae), Herpesvirus salmonis, (Herpesviridae), Herpesvirus sanguinus, (Herpesviridae), Herpesvirus scophthalmus, (Herpesviridae), Herpesvirus sylvilagus, (Herpesviridae), Herpesvirus T, (Herpesviridae), Herpesvirus tarnarinus, (Herpesviridae), Highlands J virus, (Togaviridae), Hirame rhabdovirus, (Rhabdoviridae), Hog cholera virus, (Flaviviridae), HoJo virus, (Bunyaviridae), Hepatitis delta virus, Satellites, Deltavirus, Hsiung Kaplow herpesvirus, (Herpesviridae), Hepatitis E virus, (Caliciviridae), Hepatopancreatic parvo-like virus of shrimps, (Parvoviridae), Heron hepatitis B virus, (Hepadnaviridae), Herpes ateles, (Herpesviridae), Herpes simiae virus, (Herpesviridae), Herpes simplex virus, (Herpesviridae), Herpes virus B, (Herpesviridae), Herpesvirus aotus, (Herpesviridae), Herpesvirus ateles strain, (Herpesviridae), Herpesvirus cuniculi, (Herpesviridae), Herpesvirus cyclopsis, (Herpesviridae), Huacho virus, (Reoviridae), Hughes virus, (Bunyaviridae), Human adenoviruses, (Adenoviridae), Human astrovirus, (Astroviridae), Human calicivirus, (Caliciviridae), Human caliciviruses, (Caliciviridae), Human coronavirus E, (Coronaviridae), Human coronavirus OC, (Coronaviridae), Human coxsackievirus, (Picornaviridae), Human cytomegalovirus, (Herpesviridae), Human echovirus, (Picornaviridae), Human enterovirus, (Picornaviridae), Human foamy virus, (Retroviridae), Human herpesvirus, (Herpesviridae), Human herpesvirus, Nerpesviridae, Human herpesvirus, (Herpesviridae), Human immunodeficiency virus, (Retroviridae), Human papillomavirus, (Papovaviridae), Human parainfluenza virus, (Paramyxoviridae), Human poliovirus, (Picornaviridae), Human Respiratory Syncytial Virus, (Paramyxoviridae), Human rhinovirus, (Picornaviridae), Human spumavirus, (Retroviridae), Human T-lymphotropic virus, (Retroviridae), Humpty Doo virus, (Rhabdoviridae), HV-virus, (Bunyaviridae), Hypr virus, (Flaviviridae), Laco virus, (Bunyaviridae), Ibaraki virus, (Reoviridae), Icoaraci virus, (Bunyaviridae), Ictalurid herpesvirus, (Herpesviridae), Len virus, (Reoviridae), Ife virus, (Reoviridae), Iguanid herpesvirus, (Herpesviridae), Ilesha virus, (Bunyaviridae), Ilheus virus, (Flaviviridae), Inclusion body rhinitis virus, (Herpesviridae), Infectious bovine rhinotracheitis virus, (Herpesviridae), Infectious bursal disease virus, Birnaviridae, Infectious hematopoietic necrosis virus, (Rhabdoviridae), Infectious laryngotracheitis virus, (Herpesviridae), Infectious pancreatic necrosis virus, Birnavirzdae, InfluenzaA virus (A/PR//(HN), (Orthomyxoviridae), Influenza B virus (B/Lee/), (Orthomyxoviridae), Influenza C virus (C/California/), (Orthomyxoviridae), Ingwavuma virus, (Bunyaviridae), Inini virus, (Bunyaviridae), Inkoo virus, (Bunyaviridae), Inner Frame virus, (Reoviridae), Ippy virus, (Arenaviridae), Irituia virus, (Reoviridae), Isfahan virus, (Rhabdoviridae), Israel turkey meningoencephalitis virus, (Flaviviridae), Issyk-Kul virus, (Bunyaviridae), Itaituba virus, (Bunyaviridae), Itaporanga virus, (Bunyaviridae), Itaqui virus, (Bunyaviridae), Itimirirn virus, (Bunyaviridae), Itupiranga virus, (Reoviridae), Jaagsiekte virus, (Retroviridae), Jacareacanga virus, (Reoviridae), Jamanxi virus, (Reoviridae), Jamestown Canyon virus, (Bunyaviridae), Japanaut virus, (Reoviridae), Japanese encephalitis virus, (Flaviviridae), Jan virus, (Reoviridae), JC virus, (Papovaviridae), Joa virus, (Bunyaviridae), Joinjakaka virus, (Rhabdoviridae), Juan Diaz virus, (Bunyaviridae), Jugra virus, (Flaviviridae), Juncopox virus, (Poxviridae), Junin virus, (Arenaviridae), Junonia coenia densovirus, (Parvoviridae), Jurona virus, (Rhabdoviridae), Jutiapa virus, (Flaviviridae), K virus, (Papovaviridae), K virus, (Bunyaviridae), Kachemak Bay virus, (Bunyaviridae), Kadarn virus, (Flaviviridae), Kaeng Khoi virus, (Bunyaviridae), Kaikalur virus, (Bunyaviridae), Kairi virus, (Bunyaviridae), Kaisodi virus, (Bunyaviridae), Kala Iris virus, (Reoviridae), Kamese virus, (Rhabdoviridae), Karnmavanpettai virus, (Reoviridae), Kannamangalam virus, (Rhabdoviridae), Kao Shuan virus, (Bunyaviridae), Karimabad virus, (Bunyaviridae), Karshi virus, (Flaviviridae), Kasba virus, (Reoviridae), Kasokero virus, (Bunyaviridae), Kedougou virus, (Flaviviridae), Kemerovo virus, (Reoviridae), Kenai virus, (Reoviridae), Kennedya virus Y, Potyviridae, Kern Canyon virus, (Rhabdoviridae), Ketapang virus, (Bunyaviridae), Keterah virus, (Bunyaviridae), Keuraliba virus, (Rhabdoviridae), Keystone virus, (Bunyaviridae), Kharagysh virus, (Reoviridae), Khasan virus, (Bunyaviridae), Kilham rat virus, (Parvoviridae), Kimberley virus, (Rhabdoviridae), Kindia virus, (Reoviridae), Kinkajou herpesvirus, (Herpesviridae), Kirsten murine sarcoma virus, (Retroviridae), Kismayo virus, (Bunyaviridae), Klamath virus, (Rhabdoviridae), Kokobera virus, (Flaviviridae), Kolongo virus, (Rhabdoviridae), Koolpinyah virus, (Rhabdoviridae), Koongol virus, (Bunyaviridae), Kotonkan virus, (Rhabdoviridae), Koutango virus, (Flaviviridae), Kowanyama virus, (Bunyaviridae), Kumlinge virus, (Flaviviridae), Kunjin virus, (Flaviviridae), Kwatta virus, (Rhabdoviridae), Kyzylagach virus, (Togaviridae), La Crosse virus, (Bunyaviridae), La Joya virus, (Rhabdoviridae), La-Piedad-Michoacan-Mexico virus, (Paramyxoviridae), Lacertid herpesvirus, (Herpesviridae), Lactate dehydrogenase-elevating virus, (Arterivirus), Lagos bat virus, (Rhabdoviridae), Lake Clarendon virus, (Reoviridae), Lake Victoria cormorant herpesvirus, (Herpesviridae), Langat virus, Flaviviridae, Langur virus, (Retroviridae), Lanjan virus, (Bunyaviridae), Lapine parvovirus, (Parvoviridae), Las Maloyas virus, (Bunyaviridae), Lassa virus, (Arenaviridae), Lato river virus, (Tombusviridae), Le Dantec virus, (Rhabdoviridae), Leanyer virus, (Bunyaviridae), Lebombo virus, (Reoviridae), Lednice virus, (Bunyaviridae), Lee virus, (Bunyaviridae), Leporid herpesvirus, (Herpesviridae), Leucorrhinia dubia densovirus, (Parvoviridae), Lipovnik virus, (Reoviridae), Liverpool vervet monkey virus, (Herpesviridae), Llano Seco virus, (Reoviridae), Locusta migratona entomopoxvirus, (Poxviridae), Lokem virus, (Bunyaviridae), Lone Star virus, (Bunyaviridae), Lorisine herpesvirus, (Herpesviridae), Louping ill virus, Flaviviridae, Lucke frog herpesvirus, (Herpesviridae), Lum virus, (Parvoviridae), Lukuni virus, (Bunyaviridae), Lumpy skin disease virus, (Poxviridae), Lundy virus, (Reoviridae), Lymantria dubia densovirus, (Parvoviridae), Lymphocytic choriomeningitis virus, (Arenaviridae), Machupo virus, (Arenaviridae), Macropodid herpesvirus (Herpesviridae), Madrid virus, (Bunyaviridae), Maguari virus, (Bunyaviridae), Main Drain virus, (Bunyaviridae), Malakal virus, (Rhabdoviridae), Malignant catarrhal fever virus of European cattle, (Herpesviridae), Malpais Spring virus, (Rhabdoviridae), Malva silvestris virus, (Rhabdoviridae), Manawa virus, (Bunyaviridae), Manawatu virus, (Nodaviridae), Manitoba virus, (Rhabdoviridae), Manzanilla virus, (Bunyaviridae), Map turtle herpesvirus, (Herpesviridae), Mapputta virus, (Bunyaviridae), Maprik virus, (Bunyaviridae), Maraba virus, (Rhabdoviridae), Marburg virus, (Filoviridae), Marco virus, (Rhabdoviridae), Marek's disease herpesvirus, (Herpesviridae), Marituba virus, (Bunyaviridae), Marmodid herpesvirus, (Herpesviridae), Marmoset cytomegalovirus, (Herpesviridae), Marmoset herpesvirus, (Herpesviridae), Marmosetpox virus, (Poxviridae), Marrakai virus, (Reoviridae), Mason-Pfizer monkey virus, (Retroviridae), Masou salmon reovirus, (Reoviridae), Matruh virus, (Bunyaviridae), Matucare virus, (Reoviridae), Mayaro virus, (Togaviridae), Mboke virus, (Bunyaviridae), Meaban virus, (Flaviviridae), Measles (Edmonston) virus, (Paramyxoviridae), Medical Lake macaque herpesvirus, (Herpesviridae), Melanoplus sanguinipes entomopoxvirus, (Poxviridae), Melao virus, (Bunyaviridae), Meleagrid herpesvirus, (Herpesviridae), Melilotus latent virus, (Rhabdoviridae), Melolontha melolontha entomopoxvirus, (Poxviridae), Mengovirus, (Picornaviridae), Mermet virus, (Bunyaviridae), Mice minute virus, (Parvoviridae), Mice pneumotropic virus, (Papovaviridae), Microtus pennsylvanicus herpesvirus, (Herpesviridae), Middelburg virus, (Togaviridae), Miller's nodule virus, (Poxviridae), Mill Door virus, (Reoviridae), Minatitlan virus, (Bunyaviridae), Mink calicivirus, (Caliciviridae), Mink enteritis virus, (Parvoviridae), Minnal virus, (Reoviridae), Mirabilis mosaic virus, Caulimovirus, Mirim virus, (Bunyaviridae), Mitchell river virus, (Reoviridae), Mobala virus, (Arenaviridae), Modoc virus, (Flaviviridae), Moju virus, (Bunyaviridae), Mojui dos Campos virus, (Bunyaviridae), Mokola virus, (Rhabdoviridae), Molluscum contagiosum virus, (Poxviridae), Molluscum-likepox virus, (Poxviridae), Moloney murine sarcoma virus, (Retroviridae), Moloney virus, (Retroviridae), Monkey pox virus, (Poxviridae), Mono Lake virus, (Reoviridae), Montana myotis leukoencephalitis virus, (Flaviviridae), Monte Dourado virus, (Reoviridae), Mopeia virus, (Arenaviridae), Moriche virus, (Bunyaviridae), Mosqueiro virus, (Rhabdoviridae), Mossuril virus, (Rhabdoviridae), Mount Elgon bat virus, (Rhabdoviridae), Mouse cytomegalovirus, (Herpesviridae), Mouse Elberfield virus, (Picornaviridae), Mouse herpesvirus strain, (Herpesviridae), Mouse mammary tumor virus, (Retroviridae), Mouse thymic herpesvirus, (Herpesviridae), Movar herpesvirus, (Herpesviridae), Mucambo virus, (Togaviridae), Mudjinbarry virus, (Reoviridae), Muir Springs virus, (Rhabdoviridae), Mule deerpox virus, (Poxviridae), Multimammate mouse papillomavirus, (Papovaviridae), Mumps virus, (Paramyxoviridae), Murid herpesvirus, (Herpesviridae), Murine adenovirus, (Adenoviridae), Z murine adenovirus, (Adenoviridae), Murine hepatitis virus, (Coronaviridae), Murine herpesvirus, (Herpesviridae), Murine leukemia virus, (Retroviridae), Murine parainfluenza virus, (Paramyxoviridae), Murine poliovirus, (Picornaviridae), Murine polyomavirus, (Papovaviridae), Murray Valley encephalitis virus, (Flaviviridae), Murre virus, (Bunyaviridae), Murutucu virus, (Bunyaviridae), Mykines virus, (Reoviridae), Mynahpox virus, (Poxviridae), Myxoma virus, (Poxviridae), Nairobi sheep disease virus, (Bunyaviridae), Naranj al virus, (Flaviviridae), Nasoule virus, (Rhabdoviridae), Navarro virus, (Rhabdoviridae), Ndelle virus, (Reoviridae), Ndumu virus, (Togaviridae), Neckar river virus, (Tombusviridae), Negishi virus, (Flaviviridae), Nelson Bay virus, New Minto virus, (Rhabdoviridae), Newcastle disease virus, (Paramyxoviridae), Ngaingan virus, (Rhabdoviridae), Ngari virus, (Bunyaviridae), Ngoupe virus, (Reoviridae), Nile crocodilepox virus, (Poxviridae), Nique virus, (Bunyaviridae), Nkolbisson virus, (Rhabdoviridae), Nola virus, (Bunyaviridae), North Clett virus, (Reoviridae), North End virus, (Reoviridae), Northern cereal mosaic virus, (Rhabdoviridae), Northern pike herpesvirus, (Herpesviridae), Northway virus, (Bunyaviridae), NorwaLk virus, (Caliciviridae), Ntaya virus, (Flaviviridae), Nugget virus, (Reoviridae), Nyabira virus, (Reoviridae), Nyamanini virus, Unassigned, Nyando virus, (Bunyaviridae), Oak-Vale virus, (Rhabdoviridae), Obodhiang virus, (Rhabdoviridae), Oceanside virus, (Bunyaviridae), Ockelbo virus, (Togaviridae), Odrenisrou virus, (Bunyaviridae), Oedaleus senegalensis entomopoxvirus, (Poxviridae), Oita virus, (Rhabdoviridae), Okhotskiy virus, (Reoviridae), Okola virus, (Bunyaviridae), Olifantsvlei virus, (Bunyaviridae), Omo virus, (Bunyaviridae), Omsk hemorrhagic fever virus, (Flaviviridae), Onchorhynchus masou herpesvirus, (Herpesviridae), O'nyong-nyong virus, (Togaviridae), Operophtera brurnata entomopoxvirus, (Poxviridae), Orangutan herpesvirus, (Herpesviridae), Orf virus, (Poxviridae), Oriboca virus, (Bunyaviridae), Oriximina virus, (Bunyaviridae), Oropouche virus, (Bunyaviridae), Orungo virus, (Reoviridae), Oryctes rhinoceros virus, Unassigned, Ossa virus, (Bunyaviridae), Ouango virus, (Rhabdoviridae), Oubi virus, (Bunyaviridae), Ourem virus, (Reoviridae), Ovine adeno-associated virus, (Parvoviridae), Ovine adenoviruses, (Adenoviridae), (Astroviridae), Ovine herpesvirus, (Herpesviridae), Ovine pulmonary adenocarcinoma virus, (Retroviridae), Owl hepatosplenitis herpesvirus, (Herpesviridae), P virus, (Bunyaviridae), Pacheco's disease virus, (Herpesviridae), Pacora virus, (Bunyaviridae), Pacui virus, (Bunyaviridae), Pahayokee virus, (Bunyaviridae), Palestina virus, (Bunyaviridae), Palyam virus, (Reoviridae), Pan herpesvirus, (Herpesviridae), Papio Epstein-Barr herpesvirus, (Herpesviridae), Para virus, (Bunyaviridae), Pararnushir virus, (Bunyaviridae), Parana virus, (Arenaviridae), Parapoxvirus of red deer in New Zealand, (Poxviridae), Paravaccinia virus, (Poxviridae), Parma wallaby herpesvirus, (Herpesviridae), Paroo river virus, (Reoviridae), Parrot herpesvirus, (Herpesviridae), Parry Creek virus, (Rhabdoviridae), Pata virus, (Reoviridae), Pates monkey herpesvirus pH delta, (Herpesviridae), Pathum Thani virus, (Bunyaviridae), Patois virus, (Bunyaviridae), Peaton virus, (Bunyaviridae), Percid herpesvirus, (Herpesviridae), Perdicid herpesvirus, (Herpesviridae), Perinet virus, (Rhabdoviridae), Peripianata fuliginosa densovirus, (Parvoviridae), Peste-des-petits-ruminants virus, (Paramyxoviridae), Petevo virus, (Reoviridae), Phalacrocoracid herpesvirus, (Herpesviridae), Pheasant adenovirus, (Adenoviridae), Phnom-Penh bat virus, (Flaviviridae), Phocid herpesvirus, (Herpesviridae), Phocine (seal) distemper virus, (Paramyxoviridae), Pichinde virus, (Arenaviridae), Picola virus, (Reoviridae), Pieris rapae densovirus, (Parvoviridae), Pigeon herpesvirus, (Herpesviridae), Pigeonpox virus, (Poxviridae), Badnavirus Piry virus, (Rhabdoviridae), Pisum virus, (Rhabdoviridae), Pixuna virus, (Togaviridae), Playas virus, (Bunyaviridae), Pleuronectid herpesvirus, (Nerpesviridae), Pneumonia virus of mice, (Paramyxoviridae), Pongine herpesvirus, (Herpesviridae), Pongola virus, (Bunyaviridae), Ponteves virus, (Bunyaviridae), Poovoot virus, (Reoviridae), Porcine adenoviruses, (Adenoviridae), Porcine astrovirus, (Astroviridae), Porcine circovirus, Circoviridae, Porcine enteric calicivirus, (Caliciviridae), Porcine enterovirus, (Picornaviridae), Porcine epidemic diarrhea virus, (Coronaviridae), Porcine hemagglutinating encephalomyelitis virus, (Coronaviridae), Porcine parvovirus, (Parvoviridae), Porcine respiratory and reproductive syndrome, (Arterivirus), Porcine rubulavirus, (Paramyxoviridae), Porcine transmissible gastroenteritis virus, (Coronaviridae), Porcine type C oncovirus, (Retroviridae), Porton virus, (Rhabdoviridae), Potosi virus, (Bunyaviridae), Powassan virus, (Flaviviridae), Precarious Point virus, (Bunyaviridae), Pretoria virus, (Bunyaviridae), Primate calicivirus, (Caliciviridae), Prospect Hill virus, (Bunyaviridae), Pseudaletia includens densovirus, (Parvoviridae), Pseudocowpox virus, (Poxviridae), Pseudolumpy skin disease virus, (Herpesviridae), Pseudorabies virus, (Herpesviridae), Psittacid herpesvirus, (Herpesviridae), Psittacinepox virus, (Poxviridae), Puchong virus, (Rhabdoviridae), Pueblo Viejo virus, (Bunyaviridae), Puffin Island virus, (Bunyaviridae), Punta Salinas virus, (Bunyaviridae), Punta Toro virus, (Bunyaviridae), Purus virus, (Reoviridae), Puumala virus, (Bunyaviridae), Qalyub virus, (Bunyaviridae), Quailpox virus, (Poxviridae), Quokkapox virus, (Poxviridae), Rabbit coronavirus, (Coronaviridae), Rabbit fibroma virus, (Poxviridae), Rabbit hemorrhagic disease virus, (Caliciviridae), Rabbit kidney vacuolating virus, (Papovaviridae), Rabbit oral papillomavirus, (Papovaviridae), Rabbitpox virus, (Poxviridae), Rabies virus, (Rhabdoviridae), Raccoon parvovirus, (Parvoviridae), Raccoonpox virus, (Poxviridae), Radi virus, (Rhabdoviridae), Rangifer tarandus herpesvirus, (Herpesviridae), Ranid herpesvirus, (Herpesviridae), Raphanus virus, (Rhabdoviridae), Rat coronavirus, (Coronaviridae), Rat cytomegalovirus, (Herpesviridae), Rat virus, R, (Parvoviridae), Raza virus, (Bunyaviridae), Razdan virus, (Bunyaviridae), Red deer herpesvirus, (Herpesviridae), Red kangaroopox virus, (Poxviridae), Reed Ranch virus, (Rhabdoviridae), herpesvirus, (Herpesviridae), Reindeer papillomavirus, (Papovaviridae), Reptile calicivirus, (Caliciviridae), Resistencia virus, (Bunyaviridae), Restan virus, (Bunyaviridae), Reticuloendotheliosis virus, (Retroviridae), Rhesus HHV-like virus, (Herpesviridae), Rhesus leukocyte associated herpesvirus strain, (Herpesviridae), Rhesus monkey cytomegalovirus, (Herpesviridae), Rhesus monkey papillomavirus, (Papovaviridae), Rheumatoid arthritis virus, (Parvoviridae), Rift Valley fever virus, (Bunyaviridae), Rinderpest virus, (Paramyxoviridae), Rio Bravo virus, (Flaviviridae), Rio Grande virus, (Bunyaviridae), RML virus, (Bunyaviridae), Rochambeau virus, (Rhabdoviridae), Rocio virus, (Flaviviridae), Ross River virus, (Togaviridae), Rost Islands virus, (Reoviridae), Rous sarcoma virus, (Retroviridae), Royal farm virus, (Flaviuiridae), RT parvovirus, (Parvoviridae), Rubella virus, (Togaviridae), Russian spring summer encephalitis virus, (Flaviviridae), S-virus, (Reoviridae), SA virus, (Herpesviridae), Sabio virus, (Arenaviridae), Sabo virus, (Bunyaviridae), Saboya virus, (Flaviviridae), Sacbrood virus, (Picornaviridae), Sagiyama virus, (Togaviridae), Saimiriine herpesvirus, (Herpesviridae), SaintAbb's Head virus, (Reoviridae), Saint-Floris virus, (Bunyaviridae), Sakhalin virus, (Bunyaviridae), Sal Viej a virus, (Flaviviridae), Salanga virus, (Bunyaviridae), Salangapox virus, (Poxviridae), Salehabad virus, (Bunyaviridae), Salmonid herpesvirus, (Herpesviridae), Salmonis virus, (Rhabdoviridae), Sambucus vein clearing virus, (Rhabdoviridae), SanAngelo virus, (Bunyaviridae), San Juan virus, (Bunyaviridae), San Miguel sealion virus, (Caliciviridae), San Perlita virus, (Flaviviridae), Sand rat nuclear inclusion agents, (Herpesviridae), Sandfly fever Naples virus, (Bunyaviridae), Sandfly fever Sicilian virus, (Bunyaviridae), Sandjimba virus, (Rhabdoviridae), Sango virus, (Bunyaviridae), Santa Rosa virus, (Bunyaviridae), Santarem virus, (Bunyaviridae), Sapphire II virus, (Bunyaviridae), Saraca virus, (Reoviridae), Sarracenia purpurea virus, (Rhabdoviridae), Sathuperi virus, (Bunyaviridae), Saumarez Reef virus, (Flaviviridae), Sawgrass virus, (Rhabdoviridae), Schistocerca gregaria entomopoxvirus, (Poxviridae), Sciurid herpesvirus, (Herpesviridae), Sciurid herpesvirus, (Herpesviridae), Sealpox virus, (Poxviridae), Seletar virus, (Reoviridae) Semliki Forest virus, (Togaviridae), Sena Madureira virus, (Rhabdoviridae), Sendai virus, (Paramyxoviridae), Seoul Virus, (Bunyaviridae), Sepik virus, (Flaviviridae), Serra do Navio virus, (Bunyaviridae), Shamonda virus, (Bunyaviridae), Shark River virus, (Bunyaviridae), Sheep associated malignant catarrhal fever of, (Herpesviridae), Sheep papillomavirus, (Papovaviridae), Sheep pulmonary adenomatosis associated herpesvirus, (Herpesviridae), Sheeppox virus, (Poxviridae), Shiant Islands virus, (Reoviridae), Shokwe virus, (Bunyaviridae), Shope fibroma virus, (Poxviridae), Shuni virus, (Bunyaviridae), Sibine fusca densovirus, (Parvoviridae), Sigma virus, (Rhabdoviridae), Sikte water-borne virus, (Tombusviridae), Silverwater virus, (Bunyaviridae), virus, (Bunyaviridae), Simian adenoviruses, (Adenoviridae), Simian agent virus, (Papovaviridae), Simian enterovirus, (Picornaviridae), Simian foamy virus, (Retroviridae), Simian hemorrhagic fever virus, (Arterivirus), Simian hepatitis A virus, (Picornaviridae), Simian immunodeficiency virus, (Retroviridae), Simian parainfluenza virus, (Paramyxoviridae), Simian rotavirus SA, (Reoviridae), Simian sarcoma virus, (Retroviridae), Simian T-lymphotropic virus, (Retroviridae), Simian type D virus, (Retroviridae), Simian vancella herpesvirus, (Herpesviridae), Simian virus, (Papovaviridae), Simulium vittatum densovirus, (Parvoviridae), Sindbis virus, (Togaviridae), Sixgun city virus, (Reoviridae), Skunkpox virus, (Poxviridae), Smelt reovirus, (Reoviridae), Snakehead rhabdovirus, (Rhabdoviridae), Snowshoe hare virus, (Bunyaviridae), Snyder-Theilen feline sarcoma virus, (Retroviridae), Sofyn virus, (Flaviviridae), Sokoluk virus, (Flaviviridae), Soldado virus, (Bunyaviridae), Somerville virus, (Reoviridae), Sparrowpox virus, (Poxviridae), Spectacled caimanpox virus, (Poxviridae), SPH virus, (Arenaviridae), Sphenicid herpesvirus, (Herpesviridae), Spider monkey herpesvirus, (Herpesviridae), Spondweni virus, (Flaviviridae), Spring viremia of carp virus, (Rhabdoviridae), Squirrel fibroma virus, (Poxviridae), Squirrel monkey herpesvirus, (Herpesviridae), Squirrel monkey retrovirus, (Retroviridae), SR-virus, (Bunyaviridae), Sripur virus, (Rhabdoviridae), StAbbs Head virus, (Bunyaviridae), St. Louis encephalitis virus, (Flaviviridae), Starlingpox virus, (Poxviridae), Stratford virus, (Flaviviridae), Strigid herpesvirus, (Herpesviridae), Striped bass reovirus, (Reoviridae), Striped Jack nervous necrosis virus, (Nodaviridae), Stump-tailed macaque virus, (Papovaviridae), Suid herpesvirus, (Herpesviridae), Sunday Canyon virus, (Bunyaviridae), Sweetwater Branch virus, (Rhabdoviridae), Swine cytomegalovirus, (Herpesviridae), Swine infertility and respiratory syndrome virus, (Arterivirus), Swinepox virus, (Poxviridae), Tacaiuma virus, (Bunyaviridae), Tacaribe virus, (Arenaviridae), Taggart virus, (Bunyaviridae), Tahyna virus, (Bunyaviridae), Tai virus, (Bunyaviridae), Taiassui virus, (Bunyaviridae), Tamana bat virus, (Flaviviridae), Tamdy virus, (Bunyaviridae), Tamiami virus, (Arenaviridae), Tanapox virus, (Poxviridae), Tanga virus, (Bunyaviridae), Tanjong Rabok virus, (Bunyaviridae), Taro bacilliform virus, (Badnavirus), Tataguine virus, (Bunyaviridae), Taterapox virus, (Poxviridae), Tehran virus, (Bunyaviridae), Telok Forest virus, (Bunyaviridae), Tembe virus, (Reoviridae), Tembusu virus, (Flaviviridae), Tench reovirus, (Reoviridae), Tensaw virus, (Bunyaviridae), Tephrosia symptomless virus, (Tombusviridae), Termeil virus, (Bunyaviridae), Tete virus, (Bunyaviridae), Thailand virus, (Bunyaviridae), Theiler's murine encephalomyelitis virus, (Picornaviridae), Thermoproteus virus, Lipothrixviridae, Thiafora virus, (Bunyaviridae), Thimiri virus, (Bunyaviridae), Thogoto virus, (Orthomyxoviridae), Thormodseyjarklettur virus, (Reoviridae), Thottapalayam virus, (Bunyaviridae), Tibrogargan virus, (Rhabdoviridae), Tick-borne encephalitis virus, (Flaviviridae), Tillamook virus, (Bunyaviridae), Tilligerry virus, (Reoviridae), Timbo virus, (Rhabdoviridae), Tilmboteua virus, (Bunyaviridae), Tilmaroo virus, (Bunyaviridae), Tindholmur virus, (Reoviridae), Tlacotalpan virus, (Bunyaviridae), Toscana virus, (Bunyaviridae), Tradescantia/Zebrina virus, Potyviridae, Trager duck spleen necrosis virus, (Retroviridae), Tree shrew adenovirus, (Adenoviridae), Tree shrew herpesvims, (Herpesviridae), Triatoma virus, (Picornaviridae), Tribec virus, (Reoviridae), Trivittatus virus, (Bunyaviridae), Trombetas virus, (Bunyaviridae), Trubanarnan virus, (Bunyaviridae), Tsuruse virus, (Bunyaviridae), Tucunduba virus, (Bunyaviridae), Tumor virus X, (Parvoviridae), Tupaia virus, (Rhabdoviridae), Tupaiid herpesvirus, (Herpesviridae), Turbot herpesvirus, (Herpesviridae), Turbot reovirus, (Reoviridae), Turkey adenoviruses, (Adenoviridae), Turkey coronavirus, (Coronaviridae), Turkey herpesvirus, (Herpesviridae), Turkey rhinotracheitis virus, (Paramyxoviridae), Turkeypox virus, (Poxviridae), Turlock virus, (Bunyaviridae), Turuna virus, (Bunyaviridae), Tyuleniy virus, (Flaviviridae) Uasin Gishu disease virus, (Poxviridae), Uganda S virus, (Flaviviridae), Ulcerative disease rhabdovirus, (Rhabdoviridae), Umatilla virus, (Reoviridae), Umbre virus, (Bunyaviridae), Una virus, (Togaviridae), Upolu virus, (Bunyaviridae), UR sarcoma virus, (Retroviridae), Urucuri virus, (Bunyaviridae), Usutu virus, (Flaviviridae), Uting a virus, (Bunyaviridae), Utive virus, (Bunyaviridae), Uukuniemi virus, (Bunyaviridae) Vaccinia subspecies, (Poxviridae), Vaccinia virus, (Poxviridae), Vaeroy virus, (Reoviridae), Varicella-zoster virus, (Herpesviridae), Variola virus, (Poxviridae), Vellore virus, (Reoviridae), Venezuelan equine encephalitis virus, (Togaviridae), Vesicular exanthema of swine virus, (Caliciviridae), Vesicular stomatitis Alagoas virus, Rkabdoviridae, Vesicular stomatitis Indiana virus, (Rhabdoviridae), Vesicular stomatitis New Jersey virus, (Rhabdoviridae), Vilyuisk virus, (Picornaviridae), Vinces virus, (Bunyaviridae), Viper retrovirus, (Retroviridae), Viral hemorrhagic septicemia virus, (Rhabdoviridae), Virgin River virus, (Bunyaviridae), Virus III, (Herpesviridae), Visna/maedi virus, (Retroviridae), Volepoxvirus, (Poxviridae), Wad Medani virus, (Reoviridae), Wallal virus, (Reoviridae), Walleye epidermal hyperplasia, (Herpesviridae), Wanowrie virus, (Bunyaviridae), Warrego virus, (Reoviridae), Weddel water-borne virus, Tombusviridae, Weldona virus, (Bunyaviridae), Wesselsbron virus, (Flaviviridae), West Nile virus, (Flaviviridae), Western equine encephalitis virus, (Togaviridae), Wexford virus, (Reoviridae), Whataroa virus, (Togaviridae), Wildbeest herpesvirus, (Herpesviridae), Witwatersrand virus, (Bunyaviridae), Wongal virus, (Bunyaviridae), Wongorr virus, (Reoviridae), Woodchuck hepatitis B virus, (Hepadnaviridae), Woodchuck herpesvirus marmota, (Herpesviridae), Woolly monkey sarcoma virus, (Retroviridae), Wound tumor virus, (Reoviridae), WVU virus, (Reoviridae), WW virus, (Reoviridae), Wyeomyia virus, (Bunyaviridae), Xiburema virus, (Rhabdoviridae), Xingu virus, (Bunyaviridae), Y sarcoma virus, (Retroviridae), Yaba monkey tumor virus, (Poxviridae), Yaba-virus, (Bunyaviridae), Yaba-virus, (Bunyaviridae), Yacaaba virus, (Bunyaviridae), Yaounde virus, (Flaviviridae), Yaquina Head virus, (Reoviridae), Yata virus, (Rhabdoviridae), Yellow fever virus, (Flaviviridae), Yogue virus, (Bunyaviridae), Yokapox virus, (Poxviridae), Yokase virus, (Flaviviridae), Yucca baciliform virus, Badnavirus, Yug Bogdanovac virus, (Rhabdoviridae), Zaliv Terpeniya virus, (Bunyaviridae), Zea mays virus, (Rhabdoviridae), Zegla virus, (Bunyaviridae), Zika virus, (Flaviviridae), Zirqa virus, (Bunyaviridae).

### Fungal Pathogens

Pathogenic fungi are fungi that cause disease in humans or other organisms. The pathogenic fungi which may be prevented and/or treated by the compositions and methods of the invention include but are not limited to the following.

Candida species are important human pathogens that are best known for causing opportunist infections in immunocompromised hosts (e.g., transplant patients, AIDS sufferers, and cancer patients). Infections are difficult to treat and can be very serious. Aspergillus can and does cause disease in three major ways: through the production of mycotoxins; through induction of allergenic responses; and through localized or systemic infections. With the latter two categories, the immune status of the host is pivotal. The most common pathogenic species are Aspergillus fumigatus and Aspergillus flavus. Cryptococcus neoformans can cause a severe form of meningitis and meningo-encephalitis in patients with HIV infection and AIDS. The majority of Cryptococcus species lives in the soil and do not cause disease in humans. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease in non-immunocompromised people. Histoplasma capsulatum can cause histoplasmosis in humans, dogs and cats. Pneumocystis jirovecii (or Pneumocystis carinii) can cause a form of pneumonia in people with weakened immune systems, such as premature children, the elderly, transplant patients and AIDS patients. Stachybotrys chartarum or “black mold” can cause respiratory damage and severe headaches. It frequently occurs in houses in regions that are chronically damp.

Examples include Malassezia furfur; Exophiala werneckii; Microsporum species; Trichophyton species; Epidermophyton floccosum; Sporothrix schenckii; Phialophora verrucosa; Cladosporium carrinonii; Fonsecaea species; Coccidioides; Histoplasma capsulatum; Blastomyces dermatitidis; Cryptococcus neoformans; Cryptococcus gattii; Candida albicans; Aspergillus fumigatus; Aspergillus flavus; Aspergillus niger; Rhizopus; Rhizomucor; Mucor; Exserohilum.

### Parasites

Parasite presents a major health issue, particularly in under-developed countries around the world. Significant pathogenic parasites which may be prevented and/or treated by the compostions and methods of the invention include worms (roundworms, flatworms) and protozoa. Entamoeba histolytica; Giardia lamblia; Trichomonas vaginalis; Plasmodium falciparum; Plasmodium malariae; Plasmodium ovale; Plasmodium vivax; Trypanosoma cruzi; Ascaris lumbricoides; Trichinella spiralis; Toxoplasma gondii; Leishmania donovani; Leishmania tropica; Leishmania braziliensis; Schistosoma mansoni; Schistosoma japonicum; Schistosoma haematobium; Cyclospora cayetanesis; Crytosporidium, e.g., C. parvum, C. hominis; Cystoisospora species (formerly called Isospora species), e.g., C. belli; Naegleria fowleri; Acanthamoeba species; Sappinia diploidea; Sappinia pedata; Balamuthia mandrillaris; Pneumocystis jiroveci (formerly called Pneumocystis carinii); Plasmodium knowlesi; Babesia microti; Babesia divergens; Babesia duncani; Babesia (no species name yet but designated MO-1); Trypanosoma brucei rhodesiense; Trypanosoma brucei gambiense; Balantidium coli; Dientamoeba fragilis; Phylum: Microsporidia; Sarcocystis; Baylisascaris; Necator americanus; Ancylostoma duodenale; Strongloides stercoralis; Trichinella pseudospiralis; Trichinella nelsoni; Trichinella britovi; Trichinella nativa; Trichuris trichiura; Enterobius vermicularis; Anisakis simplex; Pseudoterranova decipiens; Trichostrongylus; Oesophagostomum, e.g., O. bifurcom; Angiostrongylus; Capillaria; Dirofilaria; Loa boa; Onchocerca volvulus; Wuchereria bancrofti; Brugia malayi; Brugia timori; Mansonella, M. perstans; M. streptocerca; M. ozzardi; Dracunculus mediensis; Cutaneous larva migrans (commonly Ancylostoma braziliense=dog hookworm; also A. caninum, A. ceylanicum, and Uncinaria stenocephala); Visceral larva migrans (most commonly Toxocara canis=dog roundworm, less commonly Toxocara cati=cat roundworm, Baylisascaris procyonis=raccoon roundworm) or ocular larva migrans or neural larva migrans (B. procyonis); Gnathostoma G. spinigerum and G. hispidum; Dicrocoelium dendriticum; Echinostoma, e.g., E. hortense, E. macrorchis, E. revolutum, E. ilocanu, and E. perfoliatum; Thelazia; Shistosoma japonicum; Shistosoma mansoni; Shistosoma haematobium; Shistosoma intercalatum; Shistosoma mekongi; Austrobilharzia variglandis and other schistosomes; Taenia solium; Taenia saginata; Taenia multiceps; Taenia serialis; Taenia asiatica; Diphyllobothrium latum; Hymenolepsis nana; Echinoccoccus; Paragonimus; Clonorchis sinensis; Dipylidium caninum; Fasciola, F. hepatica; F. gigantica; Fasciolopsis bush; Heterophyes heterophyes; Hymenolepsis, H. nana, H. dimnuta; Opisthorchis; Bertiella, e.g., B. studeri and B. mucronata; Macracanthorhynchus hirudinaceous; Moniliformis moniliformis; Bolbosoma species; Metagonimus yokogawai; Dioctophyme renale; Mesocestoides, e.g., M. lineatus and M. variabilis; Philophthalmus, e.g., P. lacrymosus, P. gralli, P. palpebrarum; Spirometra, e.g., S. mansoni, S. ranarum, S. mansonoides, S. erinacei; Sparganum proliferum.

### Dosage Forms

The nutritional compositions as disclosed herein can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

Tablets prepared for oral administration according to the invention, and manufactured using direct compression, will generally contain other inactive additives such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Lubricants may be present in a concentration of, for example, from about 0.25 wt. % to about 3 wt. %, 0.5 wt. % to about 2.0 wt. %, from about 0.75% to about 1.5%.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions. Disintegrants may be present in a concentration of, for example, from about 0.25 wt. % to about 3 wt. %, 0.5 wt. % to about 2.0 wt. %, from about 0.75% to about 1.5%.

Shellac, also called purified lac, a refined product obtained from the resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method as disclosed herein, the combination of the invention may be administered to mammalian species, such as dogs, cats, humans, etc. and as such may be incorporated in a conventional systemic dosage form, such as a tablet, capsule, or elixir. The above dosage forms will also include the necessary carrier material, excipient, viscosity modifier, lubricant, buffer, antibacterial, bulking agent (such as mannitol), anti-oxidants (ascorbic acid of sodium bisulfate) or the like.

The dose administered may be carefully adjusted according to age, weight and condition of the patient, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or both of the active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active substances may be administered separately in individual dosage units at the same time or carefully coordinated times.

The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, viscosity modifier, preservative, stabilizer, flavor, etc., in the particular type of unit dosage form.

### Administration

The oral, enteral, or parenteral nutritional or pharmaceutical formulation as disclosed herein may be optimized for particular types of delivery. For example, pharmaceutical or nutritional compositions for oral delivery are formulated using pharmaceutically acceptable carriers that are well known in the art. The carriers enable the agents in the composition to be formulated, for example, as a tablet, pill, capsule, solution, suspension, sustained release formulation; powder, liquid or gel for oral ingestion by the subject.

The liquid or solid compositions may contain suitable pharmaceutically acceptable excipients as set out above. Preferably the compositions are administered by the oral, intranasal or respiratory route for local or systemic effect. Compositions in preferably sterile pharmaceutically acceptable solvents may be nebulized by use of inert gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices that deliver the formulation in an appropriate manner.

Typically, the composition may be applied repeatedly for a sustained period of time topically on the part of the body to be treated, for example, the eyelids, eyebrows, skin or scalp. The dosage regimen will generally involve regular, such as daily, administration for a period of treatment of at least one month, or at least three months, or at least six months.

Alternatively, the composition may be applied intermittently, or in a pulsed manner. Accordingly, an alternative embodiment of the disclosure is to apply the composition on an intermittent or pulsed dosage schedule. For example, the composition of the disclosure may be used for two or more days, stopped, then restarted again at a time from between 2 weeks to 3 months later, and at even more long-spaced intervals in the case of the scalp.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time. Alternatively, the amount specified may be the amount administered as the average daily, average weekly, or average monthly dose.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

2 gm of Guanosine monophosphate powder was packaged in a 4″ square sachet. About a thousand sachets were manufactured. The sachet was designed so the patient will empty the powder into a glass of water or juice and swallow it. 2 gm of Guanosine monophosphate powder was packaged in a 2″ square sachet. About a thousand sachets were manufactured. The sachet was designed so the patient will empty the powder into a glass of water or juice and swallow it.

While the invention has been described in detail and with reference to specific examples thereof, it will be apparent to one skilled in the art that various changes and modifications can be made therein without departing from the spirit and scope thereof.

1. A method of preventing and/or treating a post-pathogenic infection neurological syndrome in a patient, the method comprising the steps of:
selecting a patient in need of preventing and/or treating a post-pathogenic infection neurological syndrome;
administering to the patient a nucleoside and/or nucleotide formulation which is an oral, enteral, or parenteral nutritional or pharmaceutical formulation;
wherein the post-pathogenic infection neurological syndrome is prevented and/or treated in the patient.

2. The method of claim 1 wherein the nucleoside and/or nucleotide formulation is an oral formulation.

3. The method of claim 1 wherein the nucleoside and/or nucleotide formulation is an enteral formulation.

4. The method of claim 1 wherein the nucleoside and/or nucleotide formulation is a parenteral formulation.

5. The method of claim 1 wherein the nucleoside and/or nucleotide formulation comprises nucleotides and/or nucleotide precursors selected from the group consisting of nucleosides, purine bases, pyrimidine bases, ribose, and deoxyribose.

6. The method of claim 1 wherein the nucleotide is in monophosphate, diphosphate, or triphosphate form.

7. The method of claim 1 wherein the nucleotide is a ribonucleotide or a deoxyribonucleotide.

8. The method of claim 1 wherein the nucleotides may be monomeric, dimeric, or polymeric (including RNA and DNA).

9. The method of claim 1 wherein the nucleoside and/or nucleotide may be present in the nutritional composition as a free acid or in the form of a salt, preferably a monosodium salt.

10. The method of claim 1 wherein the nucleotide is selected from the group consisting of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, inosine 5′-monophosphate, and combinations thereof.

11. The method of claim 1 wherein the nucleoside and/or nucleotide formulation is a guanosine formulation.

12. The method of claim 1 wherein the nucleoside and/or nucleotide formulation is administered to provide about 50 mg to about 999 mg of guanosine to the patient.

13. The method of claim 1 wherein the nucleoside and/or nucleotide formulation is administered in 2 or 3 divided doses.

14. The method of claim 1 wherein the nucleoside and/or nucleotide formulation is administered in doses selected from the group consisting of 250 mg, 500 mg, 1000 mg, 2000 mg, and 4000 mg.

15. The method of claim 1 wherein the nucleoside and/or nucleotide formulation is administered in single or divided doses of one to four times daily.

16. The method of claim 1 wherein the nucleoside and/or nucleotide formulation is administered multiple times per day.

17. The method of claim 1 wherein the nucleoside and/or nucleotide formulation is administered once per day, twice per day, three times per day, four times per day, or five times per day.

18. The method of claim 1 wherein the pathogenic infection is selected from the group consisting of viral infection, bacterial infection, fungal infection, parasitic infection, and combinations thereof.

19. The method of claim 1 wherein the pathogenic infection is a viral infection.

20. The method of claim 1 wherein the viral infection is selected from the group consisting of coronavirus, SARS, SARS-Cov2, Zika virus, Norovirus, Respiratory Syncytial Virus, Influenza, Adenovirus 5, HPV 11, Lassa Fever virus, Powassan virus, Rift Valley virus, West Nile virus, and combinations thereof.

21. A method of preventing and/or treating a post-pathogenic infection clotting/thrombotic disorder in a patient, the method comprising the steps of:
selecting a patient in need of preventing and/or treating a post-pathogenic infection clotting/thrombotic disorder;
administering to the patient a nucleoside and/or nucleotide formulation which is an oral, enteral, or parenteral nutritional or pharmaceutical formulation;
wherein the post-pathogenic infection clotting/thrombotic disorder is prevented and/or treated in the patient.

22. The method of claim 21 wherein the nucleoside and/or nucleotide formulation is an oral formulation.

23. The method of claim 21 wherein the nucleoside and/or nucleotide formulation is an enteral formulation.

24. The method of claim 21 wherein the nucleoside and/or nucleotide formulation is a parenteral formulation.

25. The method of claim 21 wherein the nucleoside and/or nucleotide formulation comprises nucleotides and/or nucleotide precursors selected from the group consisting of nucleosides, purine bases, pyrimidine bases, ribose, and deoxyribose.

26. The method of claim 21 wherein the nucleotide is in monophosphate, diphosphate, or triphosphate form.

27. The method of claim 21 wherein the nucleotide may be a ribonucleotide or a deoxyribonucleotide.

28. The method of claim 21 wherein the nucleotides may be monomeric, dimeric, or polymeric (including RNA and DNA).

29. The method of claim 21 wherein the nucleoside and/or nucleotide may be present in the nutritional composition as a free acid or in the form of a salt, preferably a monosodium salt.

30. The method of claim 21 wherein the nucleotide is selected from the group consisting of cytidine 5′-monophosphate, uridine 5′-monophosphate, adenosine 5′-monophosphate, guanosine 5′-1-monophosphate, and/or inosine 5′-monophosphate, more preferably cytidine 5′-monophosphate, uridine 5′-monophosphate, adenosine 5′-monophosphate, guanosine 5′-monophosphate, inosine 5′-monophosphate, and combinations thereof.

31. The method of claim 21 wherein the nucleoside and/or nucleotide formulation is a guanosine formulation.

32. The method of claim 21 wherein the nucleoside and/or nucleotide formulation is administered to provide about 50 mg to about 999 mg of guanosine to the patient.

33. The method of claim 21 wherein the nucleoside and/or nucleotide formulation is administered in 2 or 3 divided doses.

34. The method of claim 21 wherein the nucleoside and/or nucleotide formulation is administered in doses selected from the group consisting of 250 mg, 500 mg, 1000 mg, 2000 mg, and 4000 mg.

35. The method of claim 21 wherein the nucleoside and/or nucleotide formulation is administered in single or divided doses of one to four times daily.

36. The method of claim 21 wherein the nucleoside and/or nucleotide formulation is administered multiple times per day.

37. The method of claim 21 wherein the nucleoside and/or nucleotide formulation is administered once per day, twice per day, three times per day, four times per day, or five times per day.

38. The method of claim 21 wherein the pathogenic infection is selected from the group consisting of viral infection, bacterial infection, fungal infection, parasitic infection, and combinations thereof.

39. The method of claim 21 wherein the pathogenic infection is a viral infection.

40. The method of claim 21 wherein the viral infection is selected from the group consisting of coronavirus, SARS, SARS-Cov2, Zika virus, Norovirus, Respiratory Syncytial Virus, Influenza, Adenovirus 5, HPV 11, Lassa Fever virus, Powassan virus, Rift Valley virus, West Nile virus, and combinations thereof.

41. The method of claim 21 wherein said disease or disorder is a thrombotic disease or disorder and/or involves a blood clot thrombus or the potential formation of a blood clot thrombus.

42. The method of claim 21 wherein said thrombotic disease or disorder comprises acute coronary syndrome, thromboembolism, and/or thrombosis.

43. The method of claim 21 wherein said thromboembolism comprises venous thromboembolism, arterial thromboembolism, and/or cardiogenic thromboembolism.

44. The method of claim 21 wherein said venous thromboembolism comprises deep vein thrombosis and/or pulmonary embolism.

45. The method of claim 21 wherein said thrombotic disease or disorder involves dysfunctional coagulation or disseminated intravascular coagulation.

46. The method of claim 21 wherein said thrombotic disease or disorder involves a blood clot thrombus or the potential formation of a blood clot thrombus and further involves stroke and/or one or more transient ischemic attacks (TIA).

47. The method of claim 21 wherein said thrombotic disease or disorder involving a blood clot thrombus or the potential formation of a blood clot thrombus further involves stroke and wherein the subject has non-valvular atrial fibrillation.

48. The method of claim 21 wherein said thrombotic disease or disorder involving a blood clot thrombus or the potential formation of a blood clot thrombus further involves pulmonary hypertension.
